(19) United States
(12) Patent Application Publication
(10) Pub. No.: US 2023/0069370 A1
(43) Pub. Date: Mar. 2, 2023

(54) AI-ENABLED ACCESS TO HEALTHCARE SERVICES

(71) Applicant: SONY GROUP CORPORATION, TOKYO (JP)

(72) Inventors: KRISHNA PRASAD AGARA VENKATESHA RAO, BANGALORE (IN); AKSHAY SHEKHAR KADAKOL, BANGALORE (IN); SRINIDHI SRINIVASA, BANGALORE (IN); VENUGOPAL BASWARAJU, BANGALORE (IN); SANTOSH ERANIOSE, BANGALORE (IN)

(21) Appl. No.: 17/462,285

(22) Filed: Aug. 31, 2021

(51) Int. Cl. G16H 50/20; G16H 50/30; G16H 10/60; G16H 40/67; G06F 3/01; G06Q 10/10

(52) U.S. Cl. CPC G16H 50/20; G16H 50/30; G16H 10/60; G16H 40/67; G06F 3/011; G06Q 10/1095; G06Q 2220/00

(57) ABSTRACT

A system collects first data associated with user. The first data includes historical health data and set of sensor data corresponding to a set of health-monitoring parameters. The system applies first Artificial Intelligence (AI) model on the first data to compute indicators which reflect a deviation in a health condition of the user with respect to reference values. Based on the indicators, the system generates first inference data comprising labels or tags associated with a cause of the deviation. Based on the first inference data, the system determines a first requirement for which the user is required to visit a first healthcare center. The system further determines a first set of user-related data associated with the first requirement, based on the first data and the first inference data. Thereafter, the system transfers the first set of user-related data to an electronic healthcare system associated with the first healthcare center.

### REFERENCE

None.

### FIELD

Various embodiments of the disclosure relate to artificial intelligence (AI) based healthcare services. More specifically, various embodiments of the disclosure relate to a system and a method for artificial intelligence-enabled access to healthcare services.

### BACKGROUND

Advancements in the field of medical sciences have led to the development of various healthcare and medical services. Such services help patients who require urgent or non-urgent medical assistance or intervention. The healthcare and medical services may be offered to the patients through medical facilities, such as hospitals and clinics. Typically, in course of a medical consultation or treatment, a patient may visit one or more healthcare centers. Every time the patient visits a healthcare center, the patient, with a limited understanding, may explain his/her health condition, symptoms, or other relevant information to a medical practitioner. In some instances, the patient may be referred to another healthcare center for further treatment. The other healthcare center, where the patient is referred, may not have all the required information on the patient that the previous healthcare center may have. A lot of information related to the patient may be lost throughout the course of consultation. The reason behind the loss may be that patient care is mostly fragmented, and most healthcare services operate through an infrastructure (includes Information Technology (IT) infrastructure) that may be based on a closed platform. The loss of information may affect appropriate and timely delivery of medical attention or intervention to the patient.

Limitations and disadvantages of conventional and traditional approaches will become apparent to one of skill in the art, through comparison of described systems with some aspects of the present disclosure, as set forth in the remainder of the present application and with reference to the drawings.

### SUMMARY

A system and a method for artificial intelligence (AI)-enabled access to healthcare services, are provided substantially as shown in, and/or described in connection with, at least one of the figures, as set forth more completely in the claims.

These and other features and advantages of the present disclosure may be appreciated from a review of the following detailed description of the present disclosure, along with the accompanying figures in which like reference numerals refer to like parts throughout.

### BRIEF DESCRIPTION OF THE DRAWINGS

FIG. 1 is a diagram of an exemplary network environment for AI-enabled access to healthcare services, in accordance with an embodiment of the disclosure.

FIG. 2 is a diagram that illustrates an exemplary scenario for AI-enabled access to healthcare services, in accordance with an embodiment of the disclosure.

FIG. 3 is a sequence diagram that depicts a set of operations to establish an encrypted session for transfer of data associated with a user, in accordance with an embodiment of the disclosure.

FIGS. 4A and 4B collectively is a sequence diagram for a method of enabling access to services of a first healthcare center, in accordance with an embodiment of the disclosure.

FIGS. 5A and 5B collectively is a sequence diagram for method of enabling access to services of a second healthcare center, in accordance with an embodiment of the disclosure.

FIGS. 6A and 6B collectively is a sequence diagram for a method of scheduling an emergency response (ER) service, in accordance with an embodiment of the disclosure.

FIGS. 7A and 7B collectively is a sequence diagram for a method of determination of one or more recommendations of healthcare centers, in accordance with an embodiment of the disclosure.

FIG. 8 is a sequence diagram that depicts a method for a virtual reality (VR)-based consultation session, in accordance with an embodiment of the disclosure.

FIG. 9 is a diagram that depicts a master-slave configuration of a plurality of AI models, in accordance with an embodiment of the disclosure.

FIGS. 10A and 10B collectively is a sequence diagram that depicts a set of operations between the plurality of IA models, in accordance with an embodiment of the disclosure.

FIG. 11 is a diagram that depicts determination of an exemplary first requirement, by use of a first AI model, in accordance with an embodiment of the disclosure.

FIG. 12 is a block diagram of a system that enables artificial intelligence (AI)-based access to healthcare services, in accordance with an embodiment of the disclosure.

FIG. 13 is a flowchart that illustrates an exemplary method for artificial intelligence (AI)-enabled access to healthcare services, in accordance with an embodiment of the disclosure.

### DETAILED DESCRIPTION

The following described implementations may be found in the disclosed system and a method for artificial intelligence (AI)-enabled access to healthcare services. Exemplary aspects of the disclosure provide a system that may be configured to collect data associated with a user (such as a patient). The collected first data may include historical health data and a set of sensor data corresponding to a set of health-monitoring parameters. The set of sensor data may be acquired from a set of sensors, for example, a blood pressure sensor, a heart rate sensor, and a bioimpedance sensor. The set of sensors may be a part of a user device (such as a smartphone) associated with the user or may be separate from the user device.

At any time-instant, the system may apply a first AI model on the collected data to compute one or more indicators, which may reflect a deviation in a health condition of the user with respect to reference values. For example, a first indicator may indicate a value between 0 and 1, based on a confidence score of a predication of the deviation in the health condition by the first AI model. In a scenario, the system may determine the deviation in a blood pressure measurement (for example, a BP measurement of 147/92 millimeter of mercury (mm Hg)) of the user with respect to the reference value of the blood pressure measurement (such as a reference BP measurement of 120/80 mm Hg). The first indicator corresponding to the determined deviation may be 0.92. In an embodiment, the first indicator may correspond to a confidence score or a prediction score associated with the first AI model. A value closed to one may indicate that the first indicator reflects a stronger deviation in the monitored health condition of the user with respect to reference values.

Based on the computed one or more indicators, the system may generate inference data that may include one or more labels or tags associated with a cause of the deviation in the health condition. For example, the inference data may include a label of “high blood pressure”, based on the deviation in the blood pressure measurement of the user.

The system may determine based on the generated inference data, a requirement for which the user may be required to visit a healthcare center (for example, a clinic). For example, the determined requirement may be a consultation with a medical practitioner. Based on the collected data and the inference data, the system may determine a set of user-related data associated with the determined first requirement. For example, the set of user-related data may include personal details of the user (such as name, age, and gender) along with recorded sensor data (such as heart rate measurements, blood pressure measurements and blood sugar measurements) and historical health data.

The system may be configured to transfer the determined set of user-related data to an electronic healthcare system (such as a computer, a mobile device, an edge node associated with a healthcare center, or a server) associated with the healthcare center. In an embodiment, the set of user-related data may be transferred to the electronic healthcare system before or once the user visits the healthcare center. In another embodiment, the transfer may be based on a determination that the user has left for the healthcare center. The transferred set of user-related data may include all datapoints that may be required to enable a medical practitioner (who may be a doctor or nurse, for instance) at the healthcare center to analyze the health condition of the user, to diagnose any medical condition, to physically examine the user, or to prescribe or provide tests, prognosis, medicines, or intervention to the user.

In accordance with an embodiment, the system may be configured to collect medical data associated with a medical attention received by the user at the healthcare center as part of the determined requirement. The system may update the first AI model based on the collected medical data. The system may be further configured to apply the first AI model on the collected medical data and the collected data to generate inference data. Based on the generated inference data, the system may determine a requirement for which the user may be required to visit a healthcare center (such as a hospital), which may be different from the first healthcare center (such as, the clinic). For example, the second requirement may correspond to a scheduled surgery, based on the second inference data, which may include a label of “kidney stones”. In such a case, based on the collected first data, the collected medical data, and the second inference data, the system may determine a second set of user-related data. The second set of user-related data may be associated with the determined second requirement and may be required by a second electronic healthcare system (for example, a computer, a mobile device, or a server) associated with the second healthcare center. The system may transfer the determined second set of user-related data to the second electronic healthcare system. Thus, the transfer of the second set of user-related data to the second electronic healthcare system may provide the complete medical history of the user, which may include the diagnosis of the previously visited clinic, to a medical practitioner (such as, a doctor or nurse) at the second healthcare center.

In accordance with an embodiment, the system may be configured to receive, by the user device, a request to share a data portion of the collected first data with the first AI model. The system may create an encrypted session between the first AI model and the user device, based on the request. While the encrypted session is active, the system may transfer the data portion of the collected first data to first AI model and store the transferred data portion in an encrypted form on a datastore. For example, the datastore may be associated with the user device. Thus, the system may enable storage of the data portion of the collected first data in the encrypted form, and thereby preserve a privacy of the user.

The system of the present disclosure may collect all relevant datapoints, including the medical history of the user before the user seeks medical consultation and throughout the course of medical consultation. Such datapoints may be utilized by the medical practitioners of various healthcare centers (such as a primary healthcare center or a secondary healthcare center) to service all kinds of ongoing or future medical requirements of the user. The system works with a connected network of electronic healthcare systems (i.e. nodes of a distributed network), each of which may be associated with a healthcare center. An AI model may be hosted on the system and the electronic healthcare systems to analyze and exchange data on the users and medical services rendered to the users with each other.

The collected datapoints on the user may be shared with utmost privacy through encrypted sessions so that holistic information on the health condition of the user is accessible to any medical practitioner associated with any of the various healthcare centers. The system of the present disclosure may provide a user-centric (i.e., patient-centric) end-to-end solution that may monitor various health parameters of the user to determine, using an AI model, a requirement of the user for a medical consultation. The solution may also determine data that needs to be shared with the healthcare centers or an AI model associated with healthcare center before the user consults or receives services at the healthcare center. On the user front, the AI model(s) may function to assist the user in accessing various healthcare services. Whereas, on the service provider front, such model(s) may function to assist various medical practitioners in assessment of the health condition and in better understanding of the requirements of the user. At each step in the consultation process, the AI model(s) may exchange data (including collected data, learned information, and learned neural parameter values). With a distributed network, the system may not only improve a user's access to various healthcare services, but may also enable the healthcare centers to provide an improved quality of medical assistance to the user, as the system and the nodes of the network may maintain holistic information on the health condition, medical records, prescriptions, medical tests, and treatment of the user.

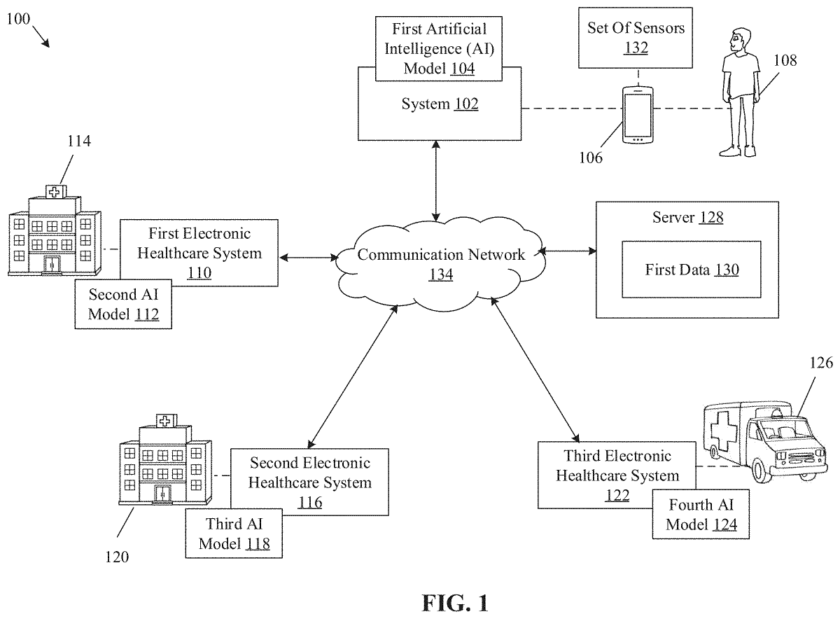

FIG. 1 is a diagram of an exemplary network environment for AI-enabled access to healthcare services, in accordance with an embodiment of the disclosure. With reference to FIG. 1, there is shown a diagram of a network environment 100. The network environment 100 may include a system 102, a first AI model 104, and a user device 106 associated with a user 108. The network environment 100 may further include a first electronic healthcare system 110 and a second AI model 112 associated with the first electronic healthcare system 110. The first electronic healthcare system 110 may be associated with a first healthcare center 114. The network environment 100 may further include a second electronic healthcare system 116 and a third AI model 118 associated with the second electronic healthcare system 116. The second electronic healthcare system 116 may be associated with a second healthcare center 120. Further, the network environment 100 may include a third electronic healthcare system 122 and a fourth AI model 124 associated with the third electronic healthcare system 122. The third electronic healthcare system 122 may be associated with an emergency response (ER) service 126. The network environment 100 may further include a server 128 on which first data 130 may be stored.

The network environment 100 may include a set of sensors 132 associated with the user device 106 and a communication network 134. The system 102, the first electronic healthcare system 110, the second electronic healthcare system 116, the third electronic healthcare system 122, and the server 128 may communicate with each other, over the communication network 134.

The system 102 may include suitable logic, circuitry, interfaces, and/or code that may be configured to apply one or more AI models to monitor the health condition of the user 108 and to determine indicators which reflect a deviation in the heath condition. While monitoring, the system 102 may determine one or more requirements for which the user 108 may be required to visit a healthcare center (such as the first healthcare center 114). The system 102 may further determine a set of user-related data associated with such requirements and may transfer the determined set of user-related data to one or more electronic healthcare systems (such as the first electronic healthcare system 110). Example implementations of the system 102 may include, but are not limited to, a cloud server (a public, private, or hybrid cloud server), a distributed computing server or a cluster of servers, a Software-as-a-Service (SaaS) application server, an edge computing system that includes a network of distributed compute/edge nodes, a mainframe system, a workstation, a personal computer, or a mobile device.

In an embodiment, the system 102 may include a frontend subsystem and a backend subsystem. The frontend subsystem may be deployed on-premises or at a location of entities, such as different healthcare centers. In an embodiment, the frontend subsystem may be a client-side application that may be accessible on user devices, such as the user device 106. The frontend subsystem may be configured to display a user interface (UI), which may include UI elements to allow the user 108 and medical practitioners to provide inputs and view the health information related to the user 108. The backend subsystem may include a server-side application, which may execute instructions related to application of AI model(s) or other operations associated with requirements of the user 108 and/or the healthcare centers.

The first AI model 104 may be a machine learning model or a deep learning model, which may be trained to identify a relationship between inputs (such as features in the form of health parameter measurements, such as a blood sugar measurement and a pulse rate measurement) of a training dataset and outputs (such as labels or scores which may be indicators or inferences associated with the health condition). The first AI model 104 may further be trained to output symptoms associated with the user 108, based on the first data 130 that the system 102 collects. The first AI model 104 may be hosted on the system 102 or on the user device 106. The first AI model 104 may be hosted on the system 102 or on the user device 106.

The first AI model 104 may be defined by a topology of network and parameters, for example, a number of weights, a cost function, an input size, a number of layers, and the like. In development of the first AI model 104, the parameters of the first AI model 104 may be tuned after every epoch of training. While training, weights may be updated so as to move towards a global minima of the cost function for the first AI model 104. After several epochs of the training on the features in the training dataset, the first AI model 104 may be trained to output a prediction/classification/regression result for a set of inputs. In case of classification, the result may be indicative of a class label for each input of the set of inputs (e.g., input features extracted from new/unseen instances).

The first AI model 104 may include electronic data, which may be implemented as, for example, a software component of an application executable on the system 102. The first AI model 104 may rely on libraries, external scripts, or other logic/instructions for execution by a processing device, such as a processor of the system 102. The first AI model 104 may include code and routines configured to enable a computing device, such as the system 102 to perform one or more operations, such as to compute one or more first indicators which may reflect the deviation in the health condition of the user 108 with respect to a reference value. Additionally, or alternatively, the first AI model 104 may be implemented using hardware including a processor, a microprocessor (e.g., to perform or control performance of one or more operations), a field-programmable gate array (FPGA), or an application-specific integrated circuit (ASIC). Alternatively, in some embodiments, the ML model may be implemented using a combination of both the hardware and the software.

In some embodiments, the first AI model 104 may be a neural network model. The neural network model may be a computational network or a system of artificial neurons or nodes, which may be arranged in a plurality of layers. The plurality of layers of the neural network model may include an input layer, one or more hidden layers, and an output layer. Each layer of the plurality of layers may include one or more nodes (or artificial neurons, represented by circles, for example). Outputs of all nodes in the input layer may be coupled to at least one node of hidden layer(s). Similarly, inputs of each hidden layer may be coupled to outputs of at least one node in other layers of the neural network model. Outputs of each hidden layer may be coupled to inputs of at least one node in other layers of the neural network model. Node(s) in the final layer may receive inputs from at least one hidden layer to output a result. The number of layers and the number of nodes in each layer may be determined from hyper-parameters of the neural network model. Such hyper-parameters may be set before, while training, or after training the neural network model on a training dataset.

Each node of the neural network model may correspond to a mathematical function (e.g., a sigmoid function or a rectified linear unit) with a set of parameters, tunable during training of the network. The set of parameters may include, for example, a weight parameter, a regularization parameter, and the like. Each node may use the mathematical function to compute an output based on one or more inputs from nodes in other layer(s) (e.g., previous layer(s)) of the neural network model. All or some of the nodes of the neural network model may correspond to same or a different mathematical function.

In training of the neural network model, one or more parameters of each node of the neural network model may be updated based on whether an output of the final layer for a given input (from the training dataset) matches a correct result based on a loss function for the neural network model. The above process may be repeated for same or a different input till a minima of loss function is achieved, and a training error is minimized. Several methods for training are known in art, for example, gradient descent, stochastic gradient descent, batch gradient descent, gradient boost, meta-heuristics, and the like.

Examples of the neural network model may include, but are not limited to, a deep neural network (DNN), a convolutional neural network (CNN), a recurrent neural network (RNN), a CNN-recurrent neural network (CNN-RNN), R-CNN, Fast R-CNN, Faster R-CNN, an artificial neural network (ANN), (You Only Look Once) YOLO network, a Long Short Term Memory (LSTM) network based RNN, CNN+ANN, LSTM+ANN, a gated recurrent unit (GRU)-based RNN, a fully connected neural network, a Connectionist Temporal Classification (CTC) based RNN, and a deep Bayesian neural network, and/or a combination of such networks. In some embodiments, the learning engine may include numerical computation techniques using data flow graphs. In certain embodiments, the neural network model may be based on a hybrid architecture of multiple Deep Neural Networks (DNNs).

The user device 106 may include suitable logic, circuitry, interfaces, and/or code that may be configured to receive a set of sensor data corresponding to a set of health-monitoring parameters associated with the user 108. The set of sensor data may be received from the set of sensors 132 associated with the user device 106. The user device 106 may include a display unit through which data such as recommendations of healthcare centers and medical practitioners may be displayed to the user 108. Examples of the user device 106 may include, but are not limited to, a wearable health device (such as a fitness band), a smartphone, a cellular phone, a mobile phone, a personal computer, a workstation, a kiosk device that may be associated with the set of sensors 132, or a consumer electronic (CE) device that may be interfaced with or associated with the set of sensors 132.

The set of sensors 132 may include suitable logic, circuitry, and/or interfaces that may be configured to monitor the set of health-monitoring parameters associated with the user 108. The set of sensors 132 may generate the set of sensor data corresponding to the monitored set of health-monitoring parameters. The set of health-monitoring parameters may include, for example, pulse rate measurements, blood pressure measurements, body temperature measurements, blood sugar measurements, one or more images of an affected portion of a body of the user 108, oxygen level measurements, pedometer measurements, breathing pattern measurements, and the like. In some embodiments, the set of sensors 132 may be communicatively coupled to the user device 106. One or more sensors of the set of sensors 132 may be integrated into the user device 106 or may be worn by the user 108. Examples of the set of sensors 132 may include, but are not limited to, a photoplethysmography (PPG) sensor, a temperature sensor, a blood pressure sensor, an ambient oxygen partial pressure (ppO2) sensor, an imaging sensor, a microphone, an artificial intelligence robot (AIBO) sensor, a step detector sensor, a step counter sensor, a glucose monitoring sensor, an accelerometer, a gyroscope, a global positioning system (GPS) sensor.

The first electronic healthcare system 110 may include suitable logic, circuitry, interfaces, and/or code that may be configured to receive a set of user-related data associated with one or more requirements (medical/health) of the user 108. The first electronic healthcare system 110 may further be configured to control a display device associated with the first healthcare center 114 to display a set of presentation data for the medical practitioner associated with the first healthcare center 114.

In some embodiments, the first electronic healthcare system 110 may host one or more AI models and may be connected to a plurality of computers or display devices at the first healthcare center 114. The first electronic healthcare system 110 may be a node of a distributed computing system that may include the system 102, the plurality of computers, or the display devices as nodes as well. Example implementations of the first electronic healthcare system 110 may include, but are not limited to, a cloud server (a public, private, or hybrid cloud server), a distributed computing server or a cluster of servers, a Software-as-a-Service (SaaS) application server, an edge computing system that includes a network of distributed compute/edge nodes), a mainframe system, a workstation, a personal computer, or a mobile device.

It should be noted that the second electronic healthcare system 116 and the third electronic healthcare system 122 may be same as or similar to the first electronic healthcare system 110, as described, for example, in FIG. 1. Therefore, the description of the second electronic healthcare system 116 and the third electronic healthcare system 122 is omitted from the disclosure for the sake of brevity.

The second AI model 112 may be associated with the first electronic healthcare system 110. In some embodiments, the second AI model 112 may be hosted on the first electronic healthcare system 110 and may be configured to interact with the first AI model 104 or the system 102 that hosts the first AI model 104, to receive information such as the first set of user-related data associated with the user 108. The second AI model 112 may also receive results or labels/tags (such as symptoms of a medical condition) that may be generated by the first AI model 104 and/or other AI models such as the third AI model 118. Based on the received results or labels/tags, the second AI model 112 may generate information that assists medical practitioners, such as nurses and doctors, to understand and diagnose issues affecting the health condition of the user 108. For example, the information may include suggestions to perform relevant medical tests for the user 108.

Architecturally, the second AI model 112, the third AI model 118, the fourth AI model 124 may be same as or similar to the first AI model 104. Therefore, the description of the third AI model 118 and the fourth AI model 124 is omitted from the disclosure for the sake of brevity. Some functions of the above models may be same and other functions may differ from each other. The difference may be based on training data on which each of such models is trained on. For example, while the first AI model 104 may monitor the health condition of the user 108, the second AI model 112 or the third AI model 118 may provide information, including insights or suggestions to medical practitioners at the healthcare centers.

The healthcare centers, such as the first healthcare center 114 and the second healthcare center 120 may correspond to entities, such as a hospital, a clinic, a medical testing lab, or a healthcare touchpoint. In some embodiments, the first healthcare center 114 may be a primary healthcare center, such as a clinic that the user 108 may visit for a primary checkup. The second healthcare center 120 may be a secondary healthcare center, such as a hospital that the user 108 may visit for any kind of medical intervention, such as a surgical procedure. In some embodiments, the first healthcare center 114 and the second healthcare center 120 may be primary healthcare centers. In some other embodiments, the first healthcare center 114 and the second healthcare center 120 may be secondary healthcare centers.

The ER service 126 may correspond to a mobile medical aid and transportation service, such as an ambulance service with/or without first responders. Examples of the ER service 126 may include, but are not limited to, a basic ambulance (for example, an ambulance with a first aid and basic life support system), an advanced ambulance (for example, an ambulance with advanced life support and intensive care system), a mortuary ambulance, and an air ambulance (which may airlift patients who may be in remote areas, critically ill, injured, or dead). Each vehicle associated with the ER service 126 may include a mobile data terminal, which may upload health condition data of the user 108 while the user 108 receives the ER service 126.

The server 128 may include suitable logic, circuitry, and interfaces, and/or code that may be configured to store data (such as the first data 130) associated with users in a secure health database, such as a HIPAA compliant database. The server 128 may be associated with the system 102 or the user device 106. The server 128 may be implemented as a cloud server and may execute operations through web applications, cloud applications, HTTP requests, repository operations, file transfer, and the like. Other example implementations of the server 128 may include, but are not limited to, a database server, a file server, a web server, a media server, an application server, a mainframe server, or a cloud computing server.

In at least one embodiment, the server 128 may be implemented as a plurality of distributed cloud-based resources by use of several technologies that are well known to those ordinarily skilled in the art. A person with ordinary skill in the art will understand that the scope of the disclosure may not be limited to the implementation of the server 128 and system 102 as two separate entities. In certain embodiments, the functionalities of the server 128 may be incorporated in its entirety or at least partially in the system 102, without departing from the scope of the disclosure.

In accordance with an embodiment, the first electronic healthcare system 110, the second electronic healthcare system 116, the third electronic healthcare system 122, the server 128, the user device 106, and the set of sensors 132 may be a part of the system 102 as nodes, communicatively coupled to each other.

In operation, the user device 106 may be configured to monitor health parameters, such as, but not limited to, the blood pressure of the user 108 and the pulse rate of the user 108. In some embodiments, the user device 106 may continuously monitor the health parameters of the user 108 when the user 108 may be at home or any other location. For example, if the user 108 is a heart patient, then the user device 106 may receive measurements related to the blood pressure, oxygen saturation, and the pulse rate of the user 108 from the set of sensors 132 at regular intervals of time (such as every minute or hour).

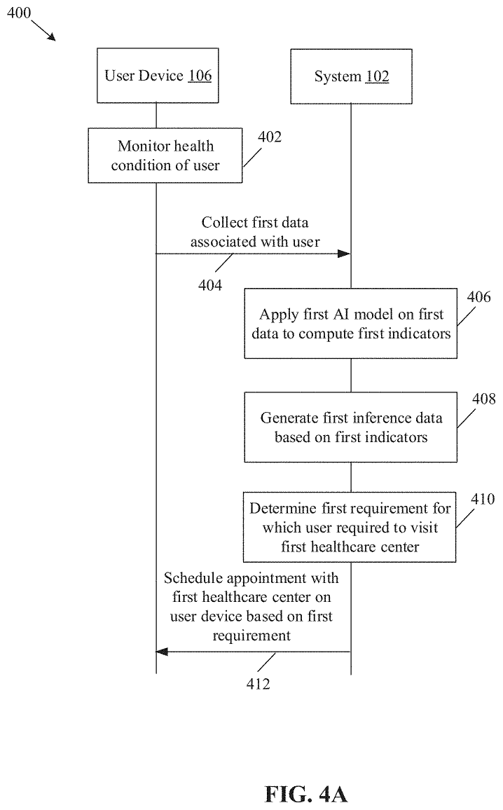

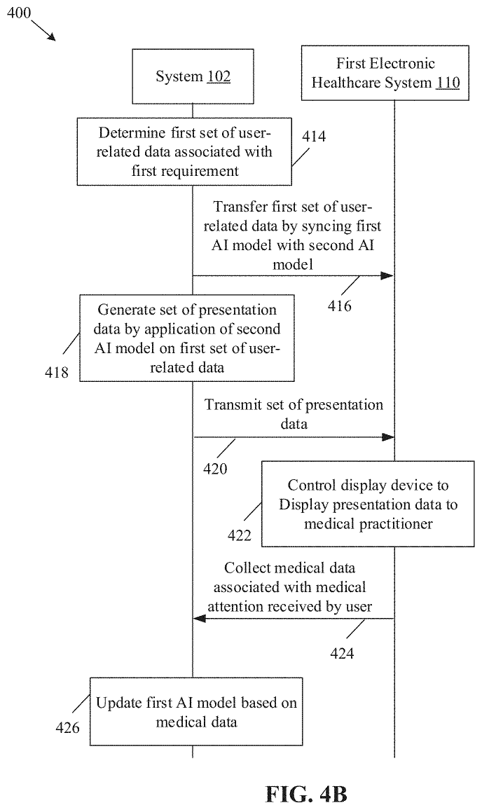

The system 102 may collect the first data 130 associated with the user 108 from the user device 106. The collected first data 130 may include historical health data and a set of sensor data corresponding to a set of health-monitoring parameters. The system 102 may collect the historical health data and the set of sensor data from the user device 106 or the server 128. The set of health-monitoring parameters may be associated with at least one of a known health condition of the user 108, one or more medical interventions that the user 108 received in past, or one or more comorbidities associated with the user 108. Additionally, or alternatively, the collected first data 130 may include past, family, and social history (PFSH) data and collaborative filtered data. The collaborative filtered data may include health-related datapoints associated with a defined population, a specific set of geographies, a specific demography, or a viral infection or outbreak within the defined population. Additionally, or alternatively, the collected first data 130 may include an appointment schedule of the user 108 for a set of medical or health interventions at one or more healthcare centers, including the first healthcare center 114. For example, the set of medical interventions may include a surgical procedure to replace a pacemaker or an implantable cardioverter defibrillator (ICD). Details of the collection of the first data 130 are further provided, for example, in FIG. 4A.

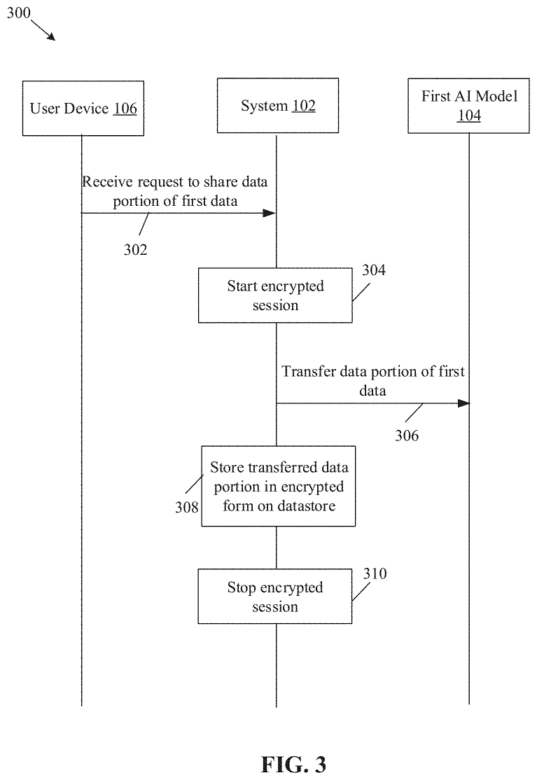

In accordance with an embodiment, the system 102 may receive, by the user device 106, a request to share a data portion of the collected first data 130 with the first AI model 104. Based on the request, the system 102 may create an encrypted session between the first AI model 104 and the user device 106. While the encrypted session is active, the system 102 may transfer the data portion of the collected first data 130 to the first AI model 104. The system 102 may store the transferred data portion in an encrypted form on a datastore. Details of the encrypted session are further provided, for example, in FIG. 3.

The system 102 may apply the first AI model 104 on the collected first data 130 to compute one or more first indicators, which may reflect a deviation in a health condition of the user 108 with respect to reference values. For example, a first indicator may include a value between 0 and 1 as a measure of a confidence of the first AI model 104 in the determination of a deviation in the health condition of the user 108. Details of the application of the first AI model 104 to compute the one or more first indicators are further provided, for example, in FIG. 4A.

Based on the computed one or more first indicators, the system 102 may generate first inference data that may include one or more labels or tags associated with a cause of the deviation in the health condition. For example, the first inference data may include labels, such as “high blood pressure”, “high blood sugar level”, “injury worsened”, and the like. Details of the generation of the first inference data are further provided, for example, in FIG. 4A.

Based on the generated first inference data, the system 102 may determine a first requirement, for which the user 108 may be required to visit the first healthcare center 114. The first requirement may correspond to, for example, a medical emergency, a scheduled medical examination, a scheduled surgical procedure, or a medical consultation. Details of the determination of the first requirement are further provided, for example, in FIG. 4A.

Based on the collected first data 130 and the first inference data, the system 102 may determine a first set of user-related data that may be associated with the determined first requirement. For example, the first requirement may be a “medical emergency”. The first set of user-related data may include contact details (such as a phone number and an address of the user 108), personal details (such as name and age, and gender of the user 108), and details associated with a cause of the medical emergency (such as a heart attack or a paralytic attack). Such details may include, for example, sensor data, biomarkers, a log of changes in health condition over a period (such as last 5 days), and historical instances of similar health conditions and medical interventions. Details of the determination of the first set of user-related data are further provided, for example, in FIG. 4B.

The system 102 may transfer the determined first set of user-related data to the first electronic healthcare system 110 associated with the first healthcare center 114. The transfer may be performed before the user 108 decides to visit the first healthcare center 114, after the user 108 books an appointment with the first healthcare center 114, or while the user 108 is on the way to the first healthcare center 114. Details of the transfer of the determined first set of user-related data are further provided, for example, in FIG. 4B.

In some embodiments, the first AI model 104 may output symptoms associated with a current health condition of the user 108, based on the received data portion of the collected first data 130. The first AI model 104 may interact with the second AI model 112 to transfer the symptoms to the second AI model 112. At the first healthcare center 114, the second AI model 112 may suggest relevant tests associated with the symptoms to one or more medical practitioners.

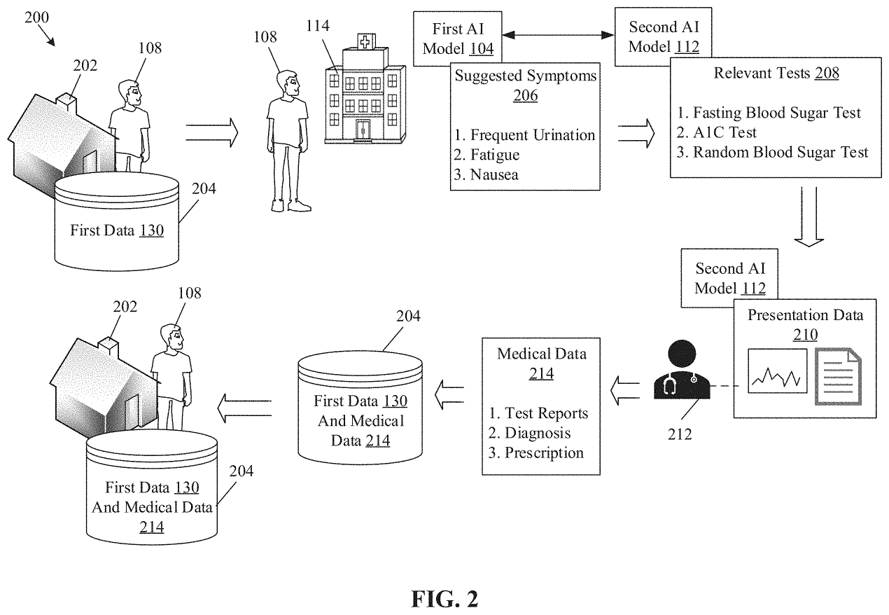

In accordance with an embodiment, the system 102 may generate a set of presentation data based on an application of the second AI model 112 on the transferred first set of user-related data. The set of presentation data may include datapoints which may be required by a medical practitioner, such as a doctor or a nurse associated with the first healthcare center 114 to assess a current health condition of the user 108 and to service the determined first requirement. The system 102 may control a display device (for example, a display device associated with the first electronic healthcare system 110) to display the generated set of presentation data for the medical practitioner. Details of the generation of the set of presentation data are further provided, for example, in FIGS. 2 and 4B.

In accordance with an embodiment, the system 102 may detect a presence of the user 108 at the first healthcare center 114. Based on the detection, the system 102 may collect medical data associated with a medical attention (or intervention) received by the user 108 at the first healthcare center 114, as part of the determined first requirement. The system 102 may update the first AI model 104 based on the collected medical data. Details of the update of the first AI model 104 are further provided, for example, in FIG. 4B.

In accordance with an embodiment, the system 102 may apply the first AI model 104 on the collected medical data and the collected first data 130 to generate second inference data. Based on the generated second inference data, the system 102 may determine a second requirement, for which the user 108 may be required to visit the second healthcare center 120, which may be different from the first healthcare center 114. For example, the second requirement may be a scheduled surgical procedure at a secondary healthcare center. Based on the collected first data 130, the collected medical data, and the second inference data, the system 102 may determine a second set of user-related data which may be associated with the determined second requirement and may be required by the second electronic healthcare system 116 associated with the second healthcare center 120. For example, the second set of user-related data may include the personal details, the contact details, and details of the health condition of the user 108 and of the medical attention/intervention received at the first healthcare center 114.

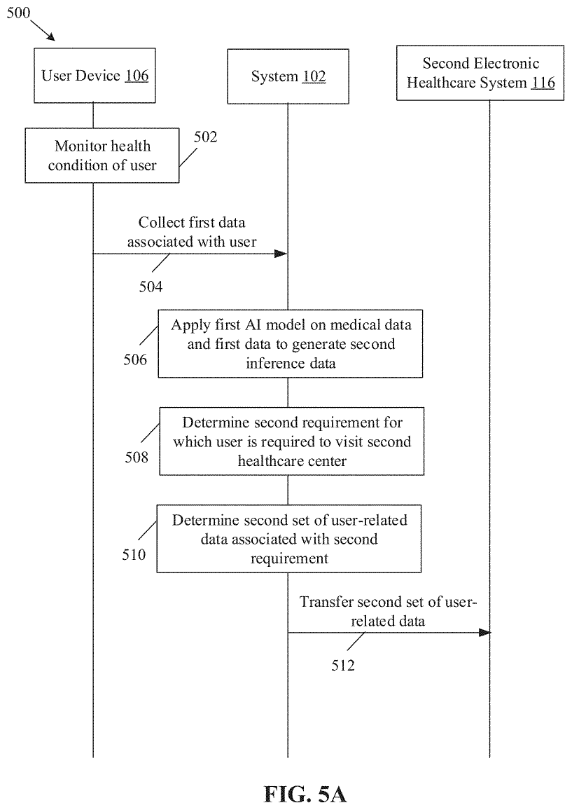

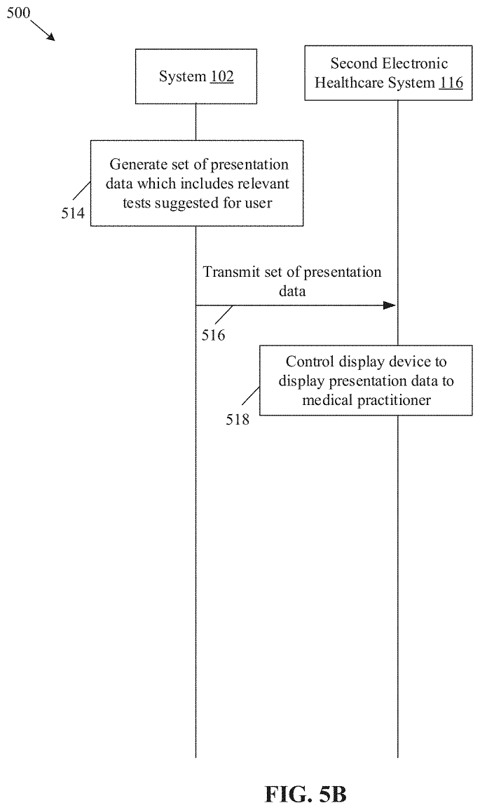

The system 102 may transfer the determined second set of user-related data to the second electronic healthcare system 116. The transfer may be performed before the user 108 decides to visit the second healthcare center 120, after the user 108 books an appointment with the second healthcare center 120, or while the user 108 is on the way to the second healthcare center 120. The second set of user-related data may be used to present data, such as insights on the health condition of the user 108 and details on a sequence of events which led the user 108 to visit the first healthcare center 114 and the second healthcare center 120. Such data may help medical practitioners at the second healthcare center 120 to provide appropriate medical attention to the user 108. Details of the medical attention that may be received by the user 108 at the second healthcare center 120 are further provided, for example, in FIGS. 5A and 5B.

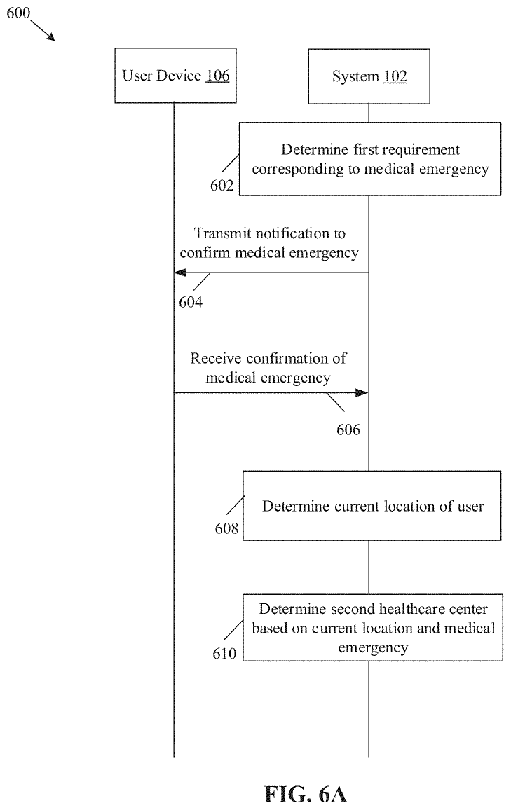

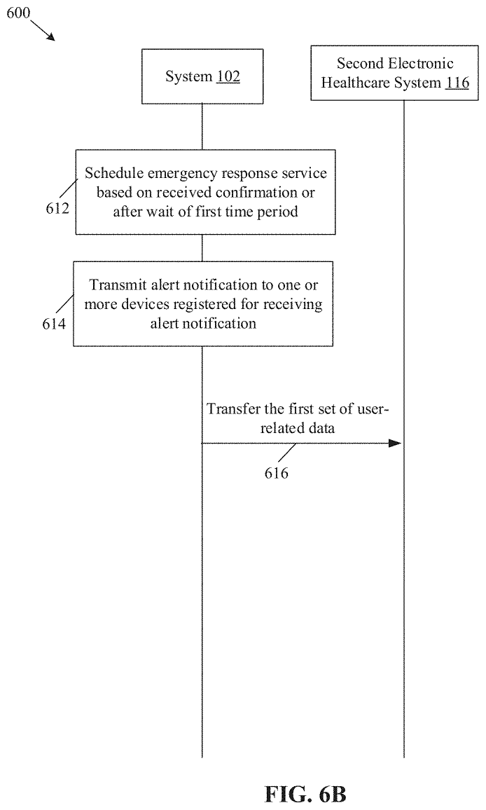

In accordance with an embodiment, the system 102 may determine the second healthcare center 120 based on a current location of the user 108 and a determination that the determined first requirement corresponds to a medical emergency. At first, the system 102 may schedule the ER service 126, such as an ambulance service to move the user 108 to the second healthcare center 120 (which may be a secondary healthcare center that may be suitable for medical emergency). Based on the scheduled ER service 126, the system 102 may transfer the first set of user-related data to the second electronic healthcare system 116 associated with the second healthcare center 120. In some embodiments, the system 102 may transfer the first set of user-related data to the third electronic healthcare system 122 associated with a third healthcare center, which may be different than the first healthcare center 114 and the second healthcare center 120. In accordance with an embodiment, the system 102 may transmit an alert notification to one or more devices (such as devices associated with family members and relatives) registered for receiving the alert notification, based on a determination that the first requirement corresponds to a medical emergency. Details of the schedule of the ER service 126 are further provided, for example, in FIGS. 6A and 6B.

In some scenarios, the user 108 may require medical assistance while the user 108 is not at home location or is travelling. In such scenarios, the system 102 may determine a current location of the user 108 and further determine, by using the first AI model 104, one or more recommendations that may include one or more healthcare centers (or touchpoints) associated with the first requirement of the user 108. The system 102 may control the user device 106 to display the determined one or more recommendations. The one or more healthcare centers may be within a threshold distance from the current location of the user 108.

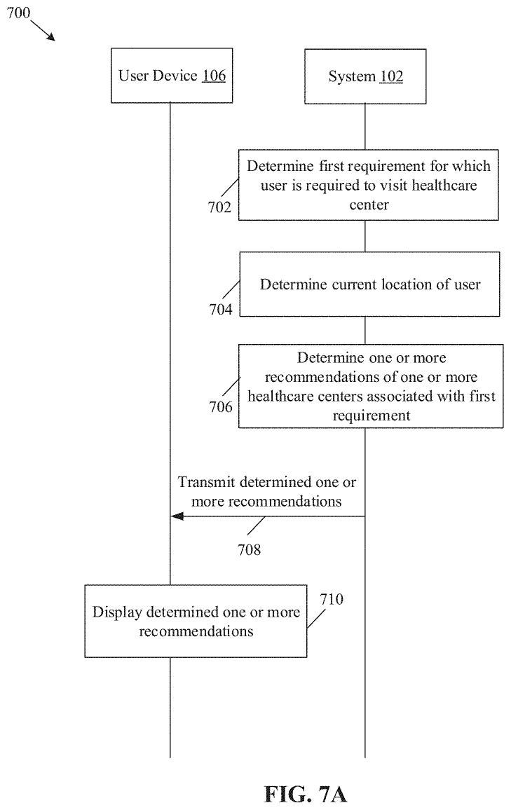

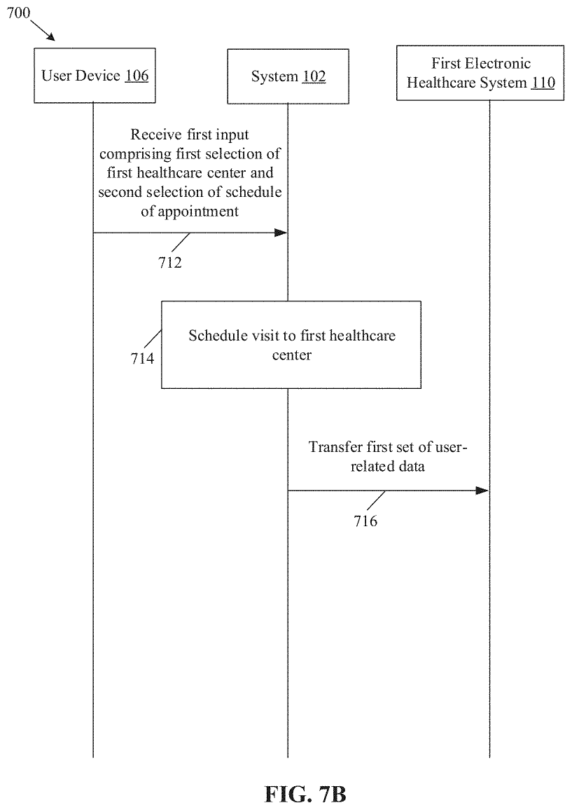

In accordance with an embodiment, the system 102 may receive a first input via the user device 106. The first input may include a first selection of the first healthcare center 114 of the one or more healthcare centers and a second selection of a schedule for an appointment with the first healthcare center 114. The system 102 may schedule a visit of the user 108 to the first healthcare center 114, based on the received first input. The first set of user-related data may be transferred to the first electronic healthcare system 110 based on the selected schedule. Details of the determination and selection of the recommendations are further provided, for example, in FIG. 7.

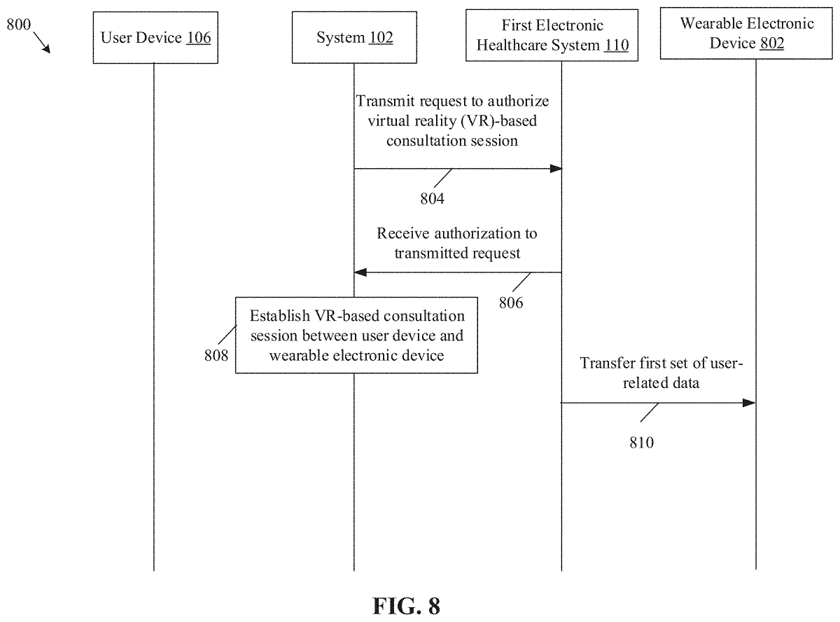

In accordance with an embodiment, the system 102 may transmit a request to the first electronic healthcare system 110 to authorize a virtual reality (VR)-based consultation session for the user 108, based on a determination that the current location of the user 108 may be different from a location of the first healthcare center 114. The system 102 may receive an authorization to the transmitted request from the first electronic healthcare system 110. Such an authorization may be provided by a medical practitioner, an admin at the first healthcare center 114, or a software which tracks availability of all medical practitioner for VR-based consultation. Based on the received authorization, the system 102 may establish the VR-based consultation session between the user device 106 and a wearable electronic device worn by the medical practitioner at the first healthcare center 114. While the VR-based consultation session may be active, the determined first set of user-related data may be transferred to the wearable electronic device and the wearable electronic device may render such data along with a video/audio/3D model feed of the user 108. Details of the VR-based consultation session are further provided, for example, in FIG. 8.

FIG. 2 is a diagram that illustrates an exemplary scenario for AI-enabled access to healthcare services, in accordance with an embodiment of the disclosure. FIG. 2 is described in conjunction with elements from FIG. 1. With reference to FIG. 2, there is shown an exemplary scenario 200. The scenario 200 may include a home 202 of the user 108, a datastore 204 associated with the system 102, the user device 106 (not shown in FIG. 2), and the server 128 (not shown in FIG. 2). It should be noted that the scenario 200 of FIG. 2 is for exemplary purpose and should not be construed for limiting the scope of the disclosure.

In the exemplary scenario 200, the user device 106 may continuously monitor the health parameters of the user 108 at the home 202 of the user 108. In some embodiments, the user device 106 may monitor the health parameters of the user 108 even when the user 108 may be outside the home 202. For example, the user device 106 may be a fitness band worn by the user 108 on a wrist of the user 108. The fitness band may monitor the health parameters, such as a pulse rate of the user 108 and a number of walking steps taken by the user 108. Further, the health parameters (such as the blood pressure measurement, the oxygen level measurements, and the blood sugar measurements) may be recorded by the user 108 or an attendant, and the recorded health parameters may be manually input to the user device 106. Alternatively, the user 108 may wear one or more sensors which may collect data on the health parameters, such as the blood pressure measurement, the oxygen level measurements, and the blood sugar measurements and may digitally upload the data to the system 102. In an embodiment, one or more images of an ailment, such as a skin infection or a wound of the user 108 may be captured by the user 108, via an imaging device (such as a camera of the user device 106 of the user 108).

The system 102 may collect the first data 130 associated with the user 108. The first data 130 may include the historical health data and the set of sensor data corresponding to the set of health-monitoring parameters. Such parameters may include the pulse rate, the number of footsteps, the blood pressure measurement, the oxygen level measurements, the blood sugar measurements, and the one or more images of an external ailment (such as a skin infection or a wound) of the user 108. The first data 130 may be stored in the datastore 204 associated with the user device 106, the system 102, or the server 128.

The system 102 may apply the first AI model 104 on the collected first data 130 of the user 108 and may compute the one or more first indicators which may reflect the deviation in the health condition of the user 108 with respect to the reference values. For example, the computed first indicator may be 0.95. The first data 130 may include the blood sugar measurements for a month-wide duration. The average of the blood sugar measurements may be 200, which may deviate with respect to the reference values (such as a value of 140). In the current scenario, the blood sugar measurements may suggest that the blood sugar level of the user 108 may be higher than the reference values. The computed first indicator may indicate an extent of deviation of the blood sugar measurement from the reference value. In an embodiment, the computed first indicator may be a confidence score associated with a prediction value of the first AI model 104, wherein the prediction value may indicate an extent of deviation of a health condition of the user 108 from reference values of the parameters associated with the health condition.

Based on the computed one or more first indicators, the system 102 may generate the first inference data, which may include the one or more labels or tags associated with the cause of the deviation. For example, the first inference data may include a label of “high blood sugar” and a cause of the deviation as “Diabetes”.

Based on the generated first inference data, the system 102 may determine the first requirement, for which the user 108 may be required to visit the first healthcare center 114. For example, the first requirement may indicate “a medical consultation for Diabetes”.

The system 102 may determine the first set of user-related data associated with the determined first requirement, based on the collected first data 130 and the first inference data. In some embodiments, whole data in the collected first data 130 and the first inference data may not be relevant for the determined first requirement. For example, the oxygen level measurements in the collected first data 130 may be irrelevant for the first requirement, such as for a medical consultation on Diabetes. The system 102 may determine the first set of user-related data that may include information relevant for diagnostics and treatment of Diabetes, such as the blood sugar measurements of the user 108, the recorded number of footsteps of the user 108, and a diet chart followed by the user 108. The first set of user-related data may further include the personal information, such as the name of the user 108, the contact details, and the address of the user 108.

The system 102 may, thereafter, transfer the determined first set of user-related data to the first electronic healthcare system 110 associated with the first healthcare center 114. In some embodiments, the first set of user-related data may be transferred to the first electronic healthcare system 110 before the user 108 reaches the first healthcare center 114. In some other embodiments, the first set of user-related data may be transferred to the first electronic healthcare system 110 once the user 108 reaches the first healthcare center 114 and upon detection of a presence of the user 108 at the first healthcare center 114.

In accordance with an embodiment, the first AI model 104 may suggest symptoms 206 that may link the current health condition of the user 108 with Diabetes. For example, the first AI model 104 may suggest the symptoms 206, such as frequent urination, fatigue, nausea, high blood sugar. In certain cases, when the user 108 is unable to explain symptoms associated with his/her medical condition, the symptoms 206 suggested by the first AI model 104 may be utilized for the diagnosis and/or treatment of the user 108.

In some embodiments, the first AI model 104 may be a part of the user device 106 associated with the user 108. Once the user 108 reaches the first healthcare center 114, the first AI model 104 may interact with the second AI model 112 hosted on the first electronic healthcare system 110 of the first healthcare center 114. Based on the interaction, the first AI model 104 may share the suggested symptoms 206 with the second AI model 112. The second AI model 112 may suggest relevant tests 208 based on the suggested symptoms 206 of the user 108. For example, the relevant tests 208 may include a fasting blood sugar test, an A1C test, and a random blood sugar test.

The second AI model 112 associated with the first electronic healthcare system 110 may generate a set of presentation data 210. The set of presentation data 210 may include the first set of user-related data (transferred by the first AI model 104), the suggested symptoms 206, and the relevant tests 208 in a structured format, based on a preference of a medical practitioner 212. The set of presentation data 210 may include, for example, prior medical records of the user 108 and a graphical representation of the health parameters (as included in the first set of user-related data), the suggested symptoms 206, and the relevant tests 208.

The set of presentation data 210 may be utilized by the medical practitioner 212, such as a nurse or a doctor, to assess the current health condition and to provide a suitable medical attention or intervention to the user 108. For example, the medical attention may include a diagnosis of the symptoms 206. Based on the diagnosis, the medical practitioner 212 may give a prescription for drugs and may suggest medical tests to the user 108. The relevant tests 208 may be referred by the medical practitioner 212 to prescribe the medical tests to the user 108.

In some embodiments, the user 108 may undergo the prescribed medical tests at the first healthcare center 114. Based on the medical assistance provided to the user 108 at the first healthcare center 114, medical data 214 associated with the user 108 may be generated. The medical data 214 may include medical test reports of the user 108, the diagnosis, and the prescription given by the medical practitioner 212. The medical data 214 may be recorded on the first electronic healthcare system 110.

The second AI model 112 may interact with the first AI model 104 to share the medical data 214 with the first AI model 104. The system 102 may update the recorded first data 130 with the medical data 214 obtained from the first healthcare center 114. The datastore 204 may now include the first data 130 as well as the medical data 214 associated with the user 108. The consultation of the user 108 may be complete based on the medical attention received by the user 108. Even when the consultation is complete, the system 102, through the user device 106, may continuously monitor the health parameters of the user 108. The system 102 may enable the healthcare centers to provide an improved quality of medical assistance to the user 108, as the system 102 may maintain holistic information of the health condition, medical records, prescriptions, medical tests, and treatment of the user 108.

FIG. 3 is a sequence diagram that depicts a set of operations to establish an encrypted session for transfer of data associated with a user, in accordance with an embodiment of the disclosure. FIG. 3 is explained in conjunction with elements from FIG. 1 and FIG. 2. With reference to FIG. 3, there is shown a sequence diagram 300 that illustrates a sequence of operations from 302 to 310. The sequence of operations may be executed by various elements of the network environment 100, such as, but not limited to, the system 102, the user device 106 and the first AI model 104.

At 302, a request to share a data portion of the collected first data 130 with the first AI model 104 may be received. In an embodiment, the system 102 may be configured to receive, by the user device 106, the request to share the data portion of the collected first data 130 with the first AI model 104. The data portion of the collected first data 130 may be shared to preserve the privacy of the user 108. The data portion of the collected first data 130 may be selected, for example, based on an input from the user 108. Any portion of the first data 130 that the user 108 doesn't prefer to share may not be included in the data portion of the collected first data 130. For example, the data portion of the collected first data 130 may exclude contact details, such as address or any medical history that the user 108 may wish to withhold.

At 304, an encrypted session between the user device 106 and the first AI model 104 may be started. In an embodiment, the system 102 may be configured to start the encrypted session between the user device 106 and the first AI model 104. The system 102 may encrypt the data portion of the collected first data 130 by utilization of a secret key. The secret key may be available on, for example, the user device 106. Upon start of the encrypted session, the secret key may be shared with the first AI model 104. The first AI model 104 may be able to access the data portion of the collected first data 130, after the first AI model 104 receives the secret key.

At 306, the data portion of the collected first data 130 may be transferred to the first AI model 104. In an embodiment, the system 102 may be configured to transfer the data portion of the collected first data 130 to the first AI model 104. As described at 304, the data portion may be encrypted and may be transferred through the encrypted session between the user device 106 and the first AI model 104. The first AI model 104 may utilize the shared data portion of the collected first data 130 to compute the one or more first indicators, which may reflect a deviation in the health condition of the user 108 with respect to the reference values.

In accordance with an embodiment, the system 102 may create the encrypted session between the first AI model 104 and the second AI model 112 associated with the first electronic healthcare system 110. Through the encrypted session, the shared data portion of the collected first data 130 and the suggested symptoms 206 may be transferred to the second AI model 112.

At 308, the transferred data portion may be stored in an encrypted form on a datastore. In an embodiment, the system 102 may be configured to store the transferred data portion in an encrypted form on the datastore 204. The system 102 may store the transferred data portion in compliance with Health Insurance Portability and Accountability Act (HIPPA) standards. With the compliance, the system 102 may ensure that the identity of the user 108 and other personally identifiable information (PII) is not accessible after the consultation has ended. Thus, the system 102 may enable complete preservation of privacy of the shared portion of the first data 130 associated with the user 108.

At 310, the encrypted session may be stopped. In an embodiment, the system 102 may be configured to stop the encrypted session. After the encrypted session ends, the system 102 may prevent a transfer of the data portion of the first data 130 to the first AI model 104. Any unauthorized entity or device may be unable to access the first data 130.

FIGS. 4A and 4B collectively is a sequence diagram for a method of enabling access to services of a first healthcare center, in accordance with an embodiment of the disclosure. FIGS. 4A and 4B are explained in conjunction with elements from FIGS. 1, 2, and 3. With reference to FIGS. 4A and 4B, there is shown a sequence diagram 400, which illustrates a sequence of operations from 402 to 426. The sequence of operations may be executed by various elements of the network environment 100, such as, but not limited to, the system 102, the user device 106, and the first electronic healthcare system 110.

At 402, the health condition of the user 108 may be monitored. In an embodiment, the user device 106 may be configured to monitor the health condition of the user 108. In an embodiment, the health condition may be monitored by configuring the set of sensors 132 associated with the user device 106 to periodically collect a set of sensor data corresponding to a set of health-monitoring parameters (such as blood pressure measurements and pulse rate measurements) of the user 108. The user device 106 may receive the set of sensor data from the set of sensors 132 at pre-determined regular intervals of time, such as every minute or hour.

In an embodiment, the user device 106, such as a smartphone of the user 108 may configure in-built set of sensors, such as, but not limited to, an accelerometer, a PPG sensor, an imaging sensor, a step detector sensor, a step counter sensor, a microphone to collect the set of sensor data.

At 404, the first data 130 associated with the user 108 may be collected. In an embodiment, the system 102 may be configured to collect the first data 130 associated with the user 108. The collected first data 130 may include historical health data and the set of sensor data corresponding to the set of health-monitoring parameters. The historical health data may include, for example, a medical history of the user 108 and recent medical interventions received by the user 108. The set of health-monitoring parameters may be associated with at least one of a known health condition of the user 108, one or more medical interventions that the user 108 received in past, or one or more comorbidities associated with the user 108. For example, the set of health-monitoring parameters may include information about known health condition, such as asthma, previous medical prescriptions, medical test reports, such as X rays and computed tomography (CT) scans, images of incurred injuries, and so forth.

In accordance with an embodiment, the first data 130 may further include past, family, and social history (PFSH) data. The past history data may include data related to one or more of past illnesses, surgical procedures, medications, or allergies of the user 108. The family history data may include data related to one or more of genetic disorders or diseases that one or more family members of the user 108 suffer from. The social history data may include data related to one or more of past and present activities (such as job and marital status) of the user 108. The first data 130 may further include collaborative filtered data that may include health-related datapoints associated with a defined population, a specific set of geographies, a specific demography, or a viral infection or outbreak within the defined population. For example, the collaborative filtered data may include data related to specific bacterial infections common in the geographical location of the user 108. In another example, the geographical location of the user 108 may be infested with mosquitoes. The collaborative filtered data may include information related to diseases (such as malaria, chikungunya, and dengue) that may be caused by certain species of the mosquitoes.

In accordance with an embodiment, the first data 130 may further include an appointment schedule for a set of medical or health interventions at one or more healthcare centers, including the first healthcare center 114. For example, the user 108 may have acute diabetes. The first data 130 may include information regarding an appointment schedule for a regular checkup for diabetes at a healthcare center, such as the first healthcare center 114.

At 406, the first AI model 104 may be applied on the collected first data 130 to compute one or more first indicators. In an embodiment, the system 102 may be configured to apply the first AI model 104 on the collected first data 130 to compute the one or more first indicators, which may reflect the deviation in the health condition of the user 108 with respect to the reference values. For example, if the user 108 is a heart patient, the computed one or more first indicators may reflect a deviation in the blood sugar measurements or cholesterol levels of the user 108. If the blood sugar measurement is “350” and the reference value of the blood sugar measurement is “140”, then the deviation may be determined as “210”, i.e., 350-140. For the above example, the first indicator may be computed as 0.97, based on the reflected deviation in the blood sugar measurement. If the value of the first indicator is close to 1 (such as 0.97), then such a value may indicate that the measured value (i.e., “350”) of the blood sugar measurement deviates to a large extent from the reference value (i.e., “140”) for the blood sugar measurement.

In an embodiment, the first indicator may correspond to a confidence score or a prediction score of the first AI model 104. More specifically, such a value may indicate a confidence of the first AI model 104 in the prediction of a deviation in blood sugar levels with respect to normal/standard blood sugar levels.

At 408, the first inference data may be generated based on the computed one or more first indicators. In an embodiment, the system 102 may be configured to generate the first inference data based on the computed one or more first indicators. The first inference data may include one or more labels or tags associated with the cause of the deviation. For example, the first inference data may include a label or a tag of “high blood sugar” as a cause of the deviation in the health condition (Diabetes).

At 410, a first requirement, for which the user 108 may be required to visit the first healthcare center 114, may be determined. In an embodiment, the system 102 may be configured to determine the first requirement, for which the user 108 may be required to visit the first healthcare center 114. The system 102 may determine the first requirement based on the generated first inference data. In some embodiments, the first requirement may correspond to one of, but not limited to, a medical emergency, a scheduled physical examination (such as a scheduled appointment), a required medical consultation, a scheduled surgical intervention, or an immediate surgical intervention. In an exemplary scenario, the system 102 may determine the cause of deviation as diabetes. In such a case, the first requirement may be a medical consultation with an Endocrinologist or a medical specialist for treatment of “diabetes”.

At 412, an appointment with the first healthcare center 114 for the user 108 may be scheduled, based on the first requirement. In an embodiment, the system 102 may be configured to schedule the appointment with the first healthcare center 114 for the user 108, based on the first requirement. The system 102 may receive a confirmation via the user device 106 of the user 108 to schedule the appointment.

In some embodiments, the system 102 may select the first healthcare center 114 based on a preference of the user 108, a current location of the user 108, and/or the first requirement. The system 102 may control the user device 106 of the user 108 to schedule the appointment with the first healthcare center 114. Thereafter, the system 102 may communicate with the first electronic healthcare system 110 of the first healthcare center 114 to schedule the appointment.

At 414, the first set of user-related data associated with the determined first requirement may be determined. The system 102 may be configured to determine the first set of user-related data based on the collected first data and the first inference data. The first set of user-related data may include the information of the user 108 that may be relevant for diagnosis and medical treatment of the user 108. For example, the first set of user-related data may include the blood sugar measurements (for example, the blood sugar measurements of a week) of the user 108, a recorded weight of the user 108, and data on any prior or existing health condition, such as a food allergy, a genetic disorder, or a hereditary disease. The first set of user-related data may further include the personal information, such as the name of the user 108, the contact details, and the address of the user 108.

At 416, the determined first set of user-related data may be transferred to the first electronic healthcare system 110 associated with the first healthcare center 114. In an embodiment, the system 102 may be configured to transfer the first set of user-related data to the first electronic healthcare system 110. In one or more embodiments, the first set of user-related data may be transferred to the first electronic healthcare system 110 before the user 108 visits the first healthcare center 114. For example, once the appointment is scheduled, the first set of user-related data may be transferred. In some embodiments, the first set of user-related data may be transferred to the first electronic healthcare system 110 once the user 108 visits the first healthcare center 114. In certain medical emergency situations, the first set of user-related data may be transferred before or while the user 108 is on the way to visit the first healthcare center 114.

In some embodiments, the system 102 may transfer the first set of user-related data based on a sync-up of the first AI model 104 with the second AI model 112 associated with the first electronic healthcare system 110. The sync-up of the first AI model 104 with the second AI model 112 may include a transfer of the first set of user-related data and weights of various nodes of the first AI model 104 to the second AI model 112. The first AI model 104 and the second AI model 112 may be re-trained based on the first set of user-related data and/or the first data 130.

At 418, the set of presentation data 210 may be generated. In accordance with an embodiment, the system 102 may be configured to generate the set of presentation data 210 by application of the second AI model 112 on the transferred first set of user-related data. The set of presentation data 210 may be generated based on a preference of the medical practitioner 212 associated with the first healthcare center 114. The set of presentation data 210 may include datapoints, which may be required by the medical practitioner 212 associated with the first healthcare center 114 to assess a current health condition of the user 108 and to service the determined first requirement.

The medical practitioner 212, for example, a doctor may require the first set of user-related data in a structured format. For example, the set of presentation data 210 may include the prior medical records sorted according to date of the medical records. The set of presentation data 210 may also include graphical representation of symptoms and health-monitoring parameters of the user 108, such as the blood sugar measurements and blood pressure measurements.

At 420, the generated set of presentation data 210 may be transferred to the first electronic healthcare system 110. In an embodiment, the system 102 may be configured to transfer the generated set of presentation data 210 to the first electronic healthcare system 110.

In another embodiment, the system 102 may transfer the generated set of presentation data 210 from the first AI model 104 to the second AI model 112. For example, to sync the first AI model 104 with the second AI model 112, the system 102 may start an encrypted session between the first AI model 104 and the second AI model 112. The sync-up of the first AI model 104 with the second AI model 112 may include a transfer of the set of presentation data 210 and weights of various nodes of the first AI model 104 to the second AI model 112. The first AI model 104 and the second AI model 112 may be re-trained based on the set of presentation data 210.

In accordance with an embodiment, the second AI model 112 may be a conversational AI hosted on the first electronic healthcare system 110 and may be associated with the first healthcare center 114. As an example, the second AI model 112 may be a chatbot, which may be accessible to the medical practitioner 212. In such a case, the second AI model 112 may be a primary AI model, and the first AI model 104 may be a secondary AI model. The medical practitioner 212 may simply have to type, select, or voice queries about the health condition of the user 108. In response, the conversational AI may generate a response to the questions that include parts of the set of presentation data 210 in a particular format. In some instances, the conversational AI may receive an audio input related to medical concern of the user 108. In response, the conversational AI may provide a transcript of the audio input in a structured manner to the medical practitioner 212.

At 422, a display device associated with the first healthcare center 114 may be controlled to display the generated set of presentation data 210. In an embodiment, the system 102 may be configured to control the display device associated with the first healthcare center 114 to display the generated set of presentation data 210. For example, the display device may be a display monitor, through which the medical practitioner 212 may view the set of presentation data 210. In another example, the display device may be associated with a user device (such as a smartphone) associated with the medical practitioner 212.

At 424, the medical data 214 associated with the medical attention received by the user 108 at the first healthcare center 114 may be collected. In an embodiment, the system 102 may be configured to collect the medical data associated with the medical attention received by the user 108 at the first healthcare center 114. The system 102 may be configured to detect a presence of the user 108 at the first healthcare center 114. Based on the detection of the presence of the user 108, the system 102 may collect the medical data 214 associated with the medical attention received by the user 108 at the first healthcare center 114, as part of the determined first requirement. In accordance with an embodiment, the system 102 may collect the medical data 214 associated with the medical attention from the first electronic healthcare system 110 associated with the first healthcare center 114. The medical data 214 may include, for example, the test reports, the diagnosis, and the prescription given by the medical practitioner 212.

At 426, the first AI model 104 may be updated based on the collected medical data 214. In an embodiment, the system 102 may be configured to update the first AI model 104 based on the collected medical data 214. In accordance with an embodiment, the first AI model 104 may sync with the second AI model 112 to update the first AI model 104. In some embodiments, the system 102 may start an encrypted session between the first AI model 104 and the second AI model 112 to enable a sync-up between the first AI model 104 and the second AI model 112. The sync-up of the first AI model 104 with the second AI model 112 may include a transfer of the collected medical data 214 and weights of various nodes of the second AI model 112 to the first AI model 104. The first AI model 104 and the second AI model 112 may be re-trained based on the collected medical data 214. In one or more embodiments, the second AI model 112 may be the primary AI model, and the first AI model 104 may be the secondary AI model.

FIGS. 5A and 5B collectively is a sequence diagram for method of enabling access to services of a second healthcare center, in accordance with an embodiment of the disclosure. FIGS. 5A and 5B are explained in conjunction with elements from FIGS. 1, 2, 3, 4A, and 4B. With reference to FIGS. 5A and 5B, there is shown a sequence diagram 500, which illustrates a sequence of operations from 502 to 518. The sequence of operations may be executed by various elements of the network environment 100, such as, but not limited to, the system 102, the user device 106, and the second electronic healthcare system 116.

At 502, the health condition of the user 108 may be monitored. In an embodiment, the user device 106 may be configured to monitor the health condition of the user 108. In an exemplary scenario, the user 108 may reach home 202 after the user 108 receives the medical treatment (based on the first requirement) from the first healthcare center 114. The user device 106 may continuously monitor the health condition of the user 108. The monitoring of the health condition of the user 108 is described, for example, at 402 of FIG. 4A.

At 504, the first data 130 associated with the user 108 may be collected. In an embodiment the system 102 may be configured to collect the first data 130 that may include the historical health data and the set of sensor data corresponding to the set of health-monitoring parameters. The collection of the first data 130 by the system 102 is described, for example, at 404 of FIG. 4A.

At 506, the first AI model 104 may be applied on the collected medical data 214 and the collected first data 130. In accordance with an embodiment, the system 102 may be configured to apply the first AI model 104 on the collected medical data 214 and the collected first data 130 to generate second inference data. In an exemplary scenario, the user 108 may be wounded. Images of the wounded portion of the body of the user 108 may be captured by the user 108 via the imaging device associated with the user device 106. The collected first data 130 may include such images of the wounded portion of the body of the user 108. The generated second inference data may include a label of “untreated wound”, based on the images of the wound in the first data 130.

At 508, a second requirement, for which the user 108 may be required to visit the second healthcare center 120, may be determined. In an embodiment, based on the generated second inference data, the system 102 may be configured to determine the second requirement, for which the user 108 may be required to visit the second healthcare center 120. The second healthcare center 120 may be different from the first healthcare center 114. For example, the second healthcare center 120 may be a secondary healthcare center, such as a hospital that may specialize in surgical procedures. The second requirement may correspond to a requirement for a surgical intervention, based on the generated second inference data.

At 510, a second set of user-related data associated with the second requirement may be determined. In an embodiment, based on the collected first data 130, the collected medical data 214, and the second inference data, the system 102 may be configured to determine the second set of user-related data associated with the determined second requirement.

The second set of user-related data may be required by the second electronic healthcare system 116 associated with the second healthcare center 120. For example, the second set of user-related data may include information about the user 108 that may be relevant for assessment of the health condition of the user 108 and for the treatment of the wound. If the user 108 is Diabetic, then the second set of user-related data may include the images of the wound, the blood sugar measurements, the blood pressure measurements, and the like.

At 512, the second set of user-related data may be transferred to the second electronic healthcare system 116. In an embodiment, the system 102 may be configured to transfer the second set of user-related data to the second electronic healthcare system 116. The second set of user-related data may be transferred to the second electronic healthcare system 116 before the user 108 visits the second electronic healthcare system 116. In some instances, the second set of user-related data may be transferred while the user 108 is on the way to visit the second healthcare center 120 or once the user 108 visits the second healthcare center 120.

In an embodiment, the system 102 may transfer the second set of user-related data based on a sync-up of the first AI model 104 with the third AI model 118 associated with the second electronic healthcare system 116. The sync-up of the first AI model 104 with the third AI model 118 may include a transfer of the second set of user-related data and weights of various nodes of the first AI model 104 to the third AI model 118. The first AI model 104 and the third AI model 118 may be re-trained based on the second set of user-related data.

At 514, the set of presentation data 210 may be generated. In an embodiment, the system 102 may be configured to generate the set of presentation data 210. In accordance with an embodiment, the set of presentation data 210 may include the second set of user-related data in a structured manner, as preferred by a medical practitioner (who may be a doctor or nurse associated with the second healthcare center 120). In another embodiment, the set of presentation data 210 may include the suggested symptoms 206 and the relevant tests 208.

At 516, the generated set of presentation data 210 may be transmitted to the second electronic healthcare system 116. In an embodiment, the system 102 may be configured to transmit the generated set of presentation data 210 to the second electronic healthcare system 116. In some embodiments, the system 102 may transfer the generated set of presentation data 210 based on a sync-up of the first AI model 104 with the third AI model 118. For example, to sync-up the first AI model 104 with the third AI model 118, the system 102 may start an encrypted session between the first AI model 104 and the third AI model 118. The sync-up of the first AI model 104 with the third AI model 118 may include a transfer of the set of presentation data 210 and weights of various nodes of the first AI model 104 to the third AI model 118. In some instances, the first AI model 104 and the third AI model 118 may be re-trained on the set of presentation data 210.

At 518, a display device associated with the second healthcare center 120 may be controlled to display the generated set of presentation data 210. In an embodiment, the system 102 may be configured to control the display device associated with the second healthcare center 120. For example, the display device may be a display monitor on which the medical practitioner may view the set of presentation data 210. In an embodiment, the system 102 may enable an encrypted session between the first AI model 104 and the third AI model 118 to update the first AI model 104 based on the medical attention received by the user 108 at the second healthcare center 120. In one or more embodiments, the third AI model 118 may be the primary AI model, and the first AI model 104 may be the secondary AI model.

Traditionally, when the user 108 visits the second healthcare center 120 after the user 108 has visited the first healthcare center 114, complete medical information associated with a treatment and health condition of the user 108, determined at the first healthcare center 114, may not be available to the second healthcare center 120. This may impact an accuracy of a diagnosis and effectiveness of a treatment that may be provided by the second healthcare center 120 to the user 108. On the other hand, the set of presentation data 210 may include the medical data 214 from the first healthcare center 114 visited by the user 108 previously. Thus, the medical practitioner of the second healthcare center 120 may be able to provide accurate diagnosis and treatment to the user 108. For example, the diagnosis and treatment of “diabetes” given by the medical practitioner 212 at the first healthcare center 114 may be utilized by the medical practitioner at the second healthcare center 120 to surgically treat the wound on the body of the user 108. As bodies of diabetic patients, such as the user 108, may require more time to heal wounds as compared to non-diabetic patients, the medical practitioner may provide more accurate diagnosis, based on the prior medical history of the user 108.

FIGS. 6A and 6B collectively is a sequence diagram for a method of scheduling an emergency response (ER) service, in accordance with an embodiment of the disclosure. FIGS. 6A and 6B are explained in conjunction with elements from FIGS. 1, 2, 3, 4A, 4B, 5A, and 5B. With reference to FIGS. 6A and 6B, there is shown a sequence diagram 600, which illustrates a sequence of operations from 602 to 616. The sequence of operations may be executed by various elements of the network environment 100, such as, but not limited to, the system 102, the user device 106, and the second electronic healthcare system 116.

At 602, the first requirement corresponding to a medical emergency may be determined. In an embodiment, the system 102 may be configured to determine the first requirement that may correspond to a medical emergency. For example, the first inference data may indicate a heart attack based on parameters, such as electrocardiogram (ECG) signal pattern and a pulse rate of the user 108. In case of a heart attack, the system 102 may determine the first requirement to be an emergency surgical procedure.

At 604, a notification to confirm the determined medical emergency may be transmitted. In an embodiment, the system 102 may be configured to transmit a notification to confirm the determined medical emergency to the user device 106. The Examples of the notification may include, but are not limited to, a pop-up alert, a text message, an alarm, or a call on the user device 106 associated with the user 108.

At 606, a confirmation of the medical emergency may be received from the user device 106. In an embodiment, the system 102 may be configured to receive the confirmation of the medical emergency from the user device 106. In case the medical emergency is determined to be inaccurate or false, the system 102 may receive an input from the user device 106 to reject the medical emergency. For example, the user 108 may have merely experienced a mild pain in the heart, which may not require an immediate medical assistance. This may help the user 108 to avoid paying any unnecessary cost related to emergency services, for example, ambulance charges.

At 608, a current location of the user 108 may be determined. In an embodiment, the system 102 may be configured to determine the current location of the user 108, based on the confirmation of the medical emergency. The system 102 may determine the current location of the user 108 by use of the set of sensor data that may include location data received from a location sensor (such as a satellite-based location receiver). In an embodiment, the confirmation received from the user device 106 may include information associated with the current location of the user 108. For example, the confirmation may include latitude and longitude co-ordinates, or GPS co-ordinates of the user device 106 associated with the user 108. The current location of the user 108 may be the home 202 of the user 108 or any other place different from the home 202. For example, the user 108 may be out of town for vacation or may be in a marketplace, his/her friend's house, or office.

At 610, the second healthcare center 120 may be determined, based on the current location of the user 108 and a determination that the first requirement corresponds to a medical emergency. In an embodiment, the system 102 may be configured to determine the second healthcare center 120, based on the current location and the medical emergency.

The system 102 may determine the second healthcare center 120 based on services that the second healthcare center 120 provides and other factors, such as a distance between the home 202 and the second healthcare center 120 and availability of medical practitioner(s) at the second healthcare center 120 to attend to the requirement of the user 108. For example, the medical emergency of heart attack may require immediate medical attention, such as surgery. The system 102 may determine a nearest healthcare center from the home 202 of the user 108 that may specialize in the heart treatment, including emergency heart procedures.

At 612, the ER service 126 may be scheduled. In an embodiment, the system 102 may schedule the ER service 126 (such as an ambulance) based on the received confirmation of the medical emergency. In an embodiment, the system 102 may wait for a defined duration starting from the time the notification is transmitted to the user device 106. If no user response or input is received in the defined duration, the system 102 may schedule the ER service 126. This may be done to provide immediate care to the user 108, in case the user 108 is incapacitated. In certain cases, the user 108 may be unresponsive, unconscious, injured, or paralyzed. In such cases, after the first defined duration (a few minutes), the system 102 may schedule the ER service 126.

At 614, an alert notification may be transmitted to one or more devices registered to receive the alert notification. In an embodiment, the system 102 may be configured to transmit the alert notification to one or more devices that may be registered to receive the alert notification. The transmission of the alert notification may be based on the determination that the first requirement corresponds to a medical emergency. For example, the one or more devices may be associated with one or more family members, relatives, friends, or acquaintances (such as, neighbors) of the user 108. The one or more devices may be mobile phones associated with the family members, relatives, friends, or acquaintances of the user 108. Examples of the alert notification may include, but are not limited to, a pop-up notification, a text message, an alarm, or a call to the one or more devices. Thus, the system 102 may provide timely alert notifications about the medical emergency of the user 108 to the family members, relatives, friends, or acquaintances of the user 108. This may enable the user 108 may receive help prompt help while the user 108 may be affected by the medical emergency.

At 616, the first set of user-related data may be transferred to the second electronic healthcare system 116. In an embodiment, the system 102 may be configured to transfer, based on the scheduled ER service 126, the first set of user-related data to the second electronic healthcare system 116 associated with the second healthcare center 120. While the ambulance may be on the way to the second healthcare center 120, the first set of user-related data may be transferred to the second electronic healthcare system 116. In some embodiments, the first AI model 104 may sync-up with the third AI model 118 to transfer the first set of user-related data. In such a case, the third AI model 118 may be the primary AI model, and the first AI model 104 may be the secondary AI model. This may enable the second healthcare center 120 to provide medical attention to the user 108 as soon as the user 108 reaches the second healthcare center 120. The first set of user-related data may include the relevant information (associated with the user 108), such as the pulse rate measurements of the user 108 and the personal details of the user 108. In some embodiments, the first set of user-related data may also include real-time or near real-time information on vital signs of the user 108.

FIGS. 7A and 7B collectively is a sequence diagram for a method of determination of one or more recommendations of healthcare centers, in accordance with an embodiment of the disclosure. FIGS. 7A and 7B are explained in conjunction with elements from FIGS. 1, 2, 3, 4A, 4B, 5A, 5B, 6A, and 6B. With reference to FIGS. 7A and 7B, there is shown a sequence diagram 700, which illustrates a sequence of operations from 702 to 716. The sequence of operations may be executed by various elements of the network environment 100, such as, but not limited to, the system 102, the user device 106, and the first electronic healthcare system 110.

At 702, the first requirement for which the user 108 may be required to visit a healthcare center may be determined. In an embodiment, the system 102 may be configured to determine the first requirement for which the user 108 may be required to visit the healthcare center. For example, the user 108 may have experienced fatigue and headache. One or more first indicators (determined by the first AI model 104) may indicate an irregular pulse rate of the user 108 and a lower blood pressure. In such a case, the first requirement may be determined as immediate medical intervention for the user 108, based on the first inference data.

At 704, the current location of the user 108 may be determined. In an embodiment, the system 102 may be configured to determine the current location of the user 108, based on the set of sensor data (included in the collected first data 130). The determination of the current location of the user 108 is described, for example, in FIG. 6. In an exemplary scenario, the current location of the user 108 may be different from that of the home 202. For example, the user 108 may be travelling or may be present in a different country, state, or district.

At 706, one or more recommendations may be determined. Such recommendations may include one or more healthcare centers associated with the determined first requirement. The one or more healthcare centers may be within a threshold distance from the current location of the user 108. For example, the one or more healthcare centers may be within a threshold distance of 500 meters from the current location of the user 108.

In accordance with an embodiment, the system 102 may be configured to determine the one or more recommendations by use of the first AI model 104. Factors that may affect the determination of the one or more recommendations may include, for example, a user preference for the healthcare center, a user preference for a medical practitioner, a history of visits by the user 108 at specific healthcare centers, a cost of consultation and treatment at one or more healthcare centers, the current location of the user 108, and the like.

At 708, the determined one or more recommendations may be transmitted to the user device 106. In an embodiment, the system 102 may be configured to transmit the determined one or more recommendations to the user device 106.

At 710, the user device 106 may be controlled to display the determined one or more recommendations. In an embodiment, the system 102 may be configured to control the user device 106 to display the determined one or more recommendations. The determined one or more recommendations may be displayed, for example, on a display screen of user's smartphone.

At 712, a first input may be received from the user device 106. In an embodiment, the system 102 may be configured to receive the first input, via the user device 106. The first input may include a first selection of the first healthcare center 114 of the one or more healthcare centers. The first input may further include a second selection of a schedule for an appointment with the first healthcare center 114. In an embodiment, the system 102 may control the user device 106 to display different slots available with the selected first healthcare center 114 for appointment. Through the first input, the user 108 may simply select a slot available with the selected first healthcare center 114. The selected slot be included in the selected schedule.

At 714, a visit to the first healthcare center 114 may be scheduled based on the received first input. In an embodiment, the system 102 may be configured to schedule the visit of the user 108 to the first healthcare center 114.

At 716, the first set of user-related data may be transferred to the first electronic healthcare system 110, based on the scheduled visit. In an embodiment, the system 102 may be configured to transfer the first set of user-related data to the first electronic healthcare system 110. The first set of user-related data may include relevant information, such as a timeseries of data on a set of health-monitoring parameters and the personal details of the user 108.

FIG. 8 is sequence diagram that depicts a method for a virtual reality (VR)-based consultation session, in accordance with an embodiment of the disclosure. FIG. 8 is explained in conjunction with elements from FIGS. 1, 2, 3, 4A, 4B, 5A, 5B, 6A, 6B, 7A, and 7B. With reference to FIG. 8, there is shown a sequence diagram 800, which illustrates a sequence of operations from 804 to 810. The sequence of operations may be executed by various elements of the network environment 100, such as, but not limited to, the system 102, the user device 106, the first electronic healthcare system 110, and a wearable electronic device 802.

At 804, a request to authorize a virtual reality (VR)-based consultation session may be transmitted to the first electronic healthcare system 110. In accordance with an embodiment, the system 102 may be configured to transmit the request to authorize the VR-based consultation session to the first electronic healthcare system 110. The request may be transmitted based on a determination that the current location of the user 108 is different from a location of the first healthcare center 114. For example, the user 108 may be at the home 202 and the user 108 may require the VR-based consultation for leg injury. In another example, the user 108 may be an old age person, for whom visiting the first healthcare center 114 may be inconvenient. Such an old age person may require the VR-based consultation for health ailments.

At 806, an authorization to the transmitted request may be received. In accordance with an embodiment, the system 102 may be configured to receive the authorization to the transmitted request from the first electronic healthcare system 110. The authorization of the first electronic healthcare system 110 for the VR-based consultation session may be required to prevent any unauthorized or malicious entity or device from connecting to the VR-based consultation session.

At 808, based on the received authorization, the VR-based consultation session may be established between the user device 106 and the wearable electronic device 802. In an embodiment, the system 102 may be configured to establish, based on the received authorization, the VR-based consultation session between the user device 106 and the wearable electronic device 802. In an embodiment, the wearable electronic device 802 may be worn by and operated by the medical practitioner 212 at the first healthcare center 114. In an embodiment, the user device 106 may include one or more imaging devices (such as camera) that may be utilized for the VR-based consultation.

At 810, the first set of user-related data may be transferred to the wearable electronic device 802. In accordance with an embodiment, the system 102 may be configured to transfer the determined first set of user-related data to the wearable electronic device 802, when the VR-based consultation session is active. The first set of user-related data may include relevant information such as images of the injury of the user 108, data on the health-monitoring parameters, and the personal details of the user 108.

The wearable electronic device 802 may be configured to receive the first set of user-related data and a video feed or a 3D avatar of the user 108. While the session is active, the wearable electronic device 802 may display the first set of user-related data through UI elements or widgets. Additionally, the wearable electronic device 802 may display the video feed (or the 3D avatar), through which the user 108 may communicate with the medical practitioner 212 or may show at least a portion of the body that requires the medical attention. For example, the wearable electronic device 802 display the injured leg of the user 108. Examples of the wearable electronic device 802 may include, but are not limited to, a smart glass, a Virtual Reality (VR)-based head-mounted device and an Augmented Reality (AR)-based head-mounted device. In some embodiments, the system 102 may establish a video consultation session as the VR-based consultation session between the user device 106 and the first electronic healthcare system 110. The wearable electronic device 802 may connect to the first electronic healthcare system 110 to provide the medical attention to the user 108.

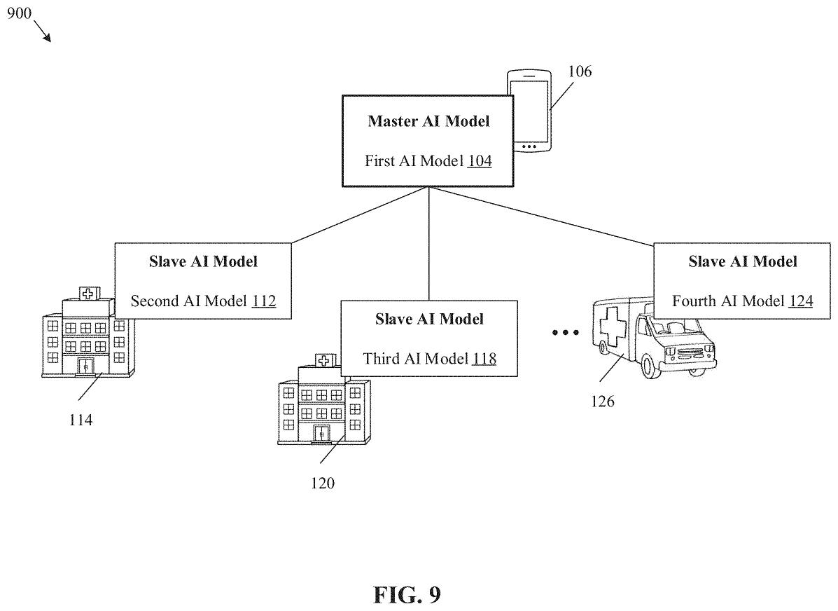

FIG. 9 is a diagram that depicts a master-slave configuration of a plurality of AI models, in accordance with an embodiment of the disclosure. FIG. 9 is explained in conjunction with elements from FIGS. 1, 2, 3, 4A, 4B, 5A, 5B, 6A, 6B, 7A, 7B, and 8. With reference to FIG. 9, there is shown a diagram 900. The diagram 900 may include the first AI model 104. The first AI model 104 may be associated with the user device 106. The diagram 900 may further include the second AI model 112 associated with the first healthcare center 114, the third AI model 118 associated with the second healthcare center 120, and the fourth AI model 124 associated with the ER service 126.

The first AI model 104, the second AI model 112, the third AI model 118, and the fourth AI model 124 may be implemented in a master-slave configuration. For example, the first AI model 104 may be a master AI model, that may be configured to control or orchestrate operations of slave AI models, such as the second AI model 112, the third AI model 118, and the fourth AI model 124 to perform the operations.

In an exemplary scenario, the first AI model 104 may transfer the first set of user-related data to the second AI model 112, based on the presence of the user 108 at the first healthcare center 114. The first AI model 104 may control the second AI model 112 to generate the set of presentation data 210. The first AI model 104 may further control the second AI model 112 to receive the medical data 214 from the second AI model 112. In an embodiment, the first AI model 104 may control the second AI model 112 to act as a chatbot, which may be accessible to the medical practitioner 212.

The first AI model 104 may transfer the second set of user-related data to the third AI model 118, based on the presence of the user 108 at the second healthcare center 120. The first AI model 104 may control the third AI model 118 to generate the set of presentation data 210 for the medical practitioner associated with the second healthcare center 120. The first AI model 104 may further control the third AI model 118 to receive the medical data 214 from the third AI model 118.

In an exemplary scenario, the system 102 may determine the first requirement corresponding to the medical emergency. Details of the determination of the first requirement corresponding to the medical emergency are further described, for example, at 604 of FIG. 6A. Based on the determined first requirement corresponding to the medical emergency, the first AI model 104 may transmit the first set of user-related data to the fourth AI model 124 associated with the ER service 126. The first AI model 104 may control the fourth AI model 124 to generate the set of presentation data 210, such that the medical practitioner may have the required set of presentation data 210 before the user 108 may reach the second healthcare center 120.

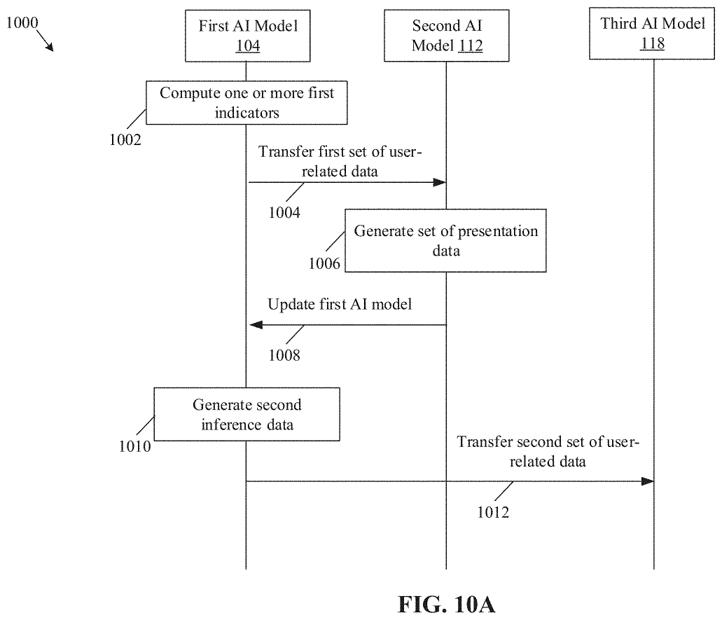

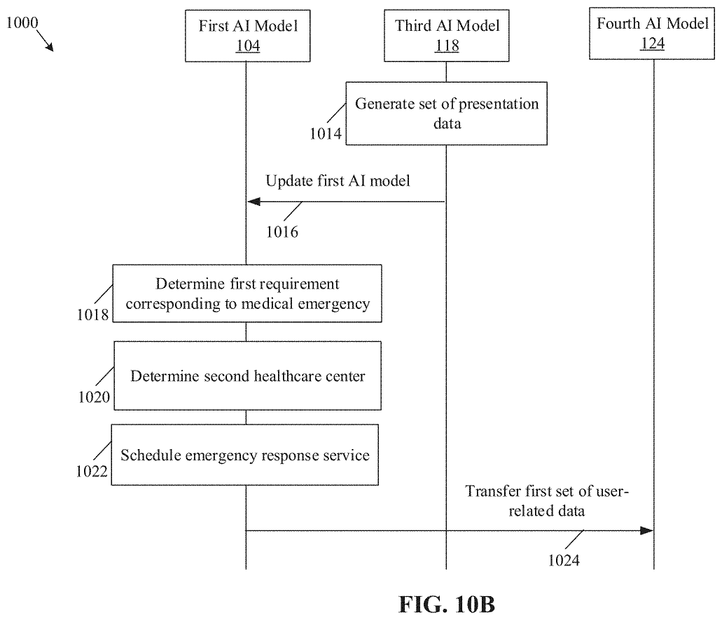

FIGS. 10A and 10B collectively is a sequence diagram that depicts a set of operations between the plurality of AI models, in accordance with an embodiment of the disclosure. FIGS. 10A and 10B are explained in conjunction with elements from FIGS. 1, 2, 3, 4A, 4B, 5A, 5B, 6A, 6B, 7A, 7B, 8 and 9. With reference to FIGS. 10A and 10B, there is shown a sequence diagram 1000, which illustrates a sequence of operations from 1002 to 1024. The sequence of operations may be executed by various entities, such as, but not limited to, the first AI model 104, the second AI model 112, the third AI model 118, and the fourth AI model 124.

At 1002, the first AI model 104 may be applied on the collected first data 130 to compute one or more first indicators. Such indicators may reflect the deviation in the health condition of the user 108 with respect to the reference values. Details of the computation of the one or more first indicators are further provided, for example, at 406 of FIG. 4A.

At 1004, the first set of user-related data may be transferred to the second AI model 112. In accordance with an embodiment, the first AI model 104 may transfer the first set of user-related data to the second AI model 112 based on a sync-up of the first AI model 104 with the second AI model 112. Details of the transfer of the first set of user-related data further provided, for example, at 416 in FIG. 4B.

At 1006, the set of presentation data 210 may be generated. In accordance with an embodiment, the second AI model 112 may be configured to generate the set of presentation data 210. For example, the first AI model 104 may control the second AI model 112 to generate the set of presentation data 210. Details of the generation of the set of presentation data 210 are further provided, for example, at 418 in FIG. 4B.

At 1008, the first AI model 104 may be updated. In accordance with an embodiment, the second AI model 112 may transmit the medical data 214 to the first AI model 104. For example, the second AI model 112 may transmit the medical data 214 to the first AI model 104 based on the sync-up of the first AI model 104 with the second AI model 112. Details of the update of the first AI model 104 are further provided, for example, at 426 in FIG. 4B.

At 1010, the second inference data may be generated. In accordance with an embodiment, the system 102 may be configured to apply the first AI model 104 on the collected medical data 214 and the collected first data 130 to generate second inference data. Details of the generation of the second inference data are further provided, for example, at 506 in FIG. 5A.

At 1012, the second set of user-related data may be transferred to the third AI model 118. The second set of user-related data may include relevant data from the first data 130 and the medical data 214 received by the first AI model 104. The second set of user-related data may be required by the second electronic healthcare system 116 associated with the second healthcare center 120. Details of the transfer of the second set of user-related data are further provided, for example, at 510 in FIG. 5A.

At 1014, the set of presentation data 210 may be generated. In accordance with an embodiment, the third AI model 118 may be configured to generate the set of presentation data 210. For example, the first AI model 104 may control the third AI model 118 to generate the set of presentation data 210. Details of the generation of the set of presentation data 210 by the third AI model 118 are further provided, for example, at 514 in FIG. 5B.

At 1016, the first AI model 104 may be updated. In accordance with an embodiment, the third AI model 118 may transmit the medical data 214 to the first AI model 104 based on a sync-up of the first AI model 104 with the third AI model 118. The medical data 214 may be generated based on a diagnosis or a treatment provided by the medical practitioner at the second healthcare center 120. The update may correspond to a process of training the first AI model 104 on the medical data 214.

At 1018, the first requirement corresponding to the medical emergency may be determined. In accordance with an embodiment, the first AI model 104 may be configured to determine the first requirement that may correspond to a medical emergency. For example, in case of a paralytic attack, the first AI model 104 may determine the first requirement to be a medical emergency.

At 1020, the second healthcare center 120 may be determined. In accordance with an embodiment, the first AI model 104 may be configured to determine the second healthcare center 120. For example, the first AI model 104 may determine the second healthcare center 120 based on the location of the user 108 and other factors, such as availability of medical practitioner(s) at the second healthcare center 120 and types of services provided at the second healthcare center 120. Details of the determination of the second healthcare center 120 are further provided, for example, at 610 in FIG. 6A.

At 1022, the ER service 126 may be scheduled. In accordance with an embodiment, the first AI model 104 may be configured to schedule the ER service 126 based on the determined medical emergency. Details of the schedule of the ER service 126 are further provided, for example, at 612 in FIG. 6B.

At 1024, the first set of user-related data may be transferred to the second AI model 112 associated with the second electronic healthcare system 116. In an embodiment, the first AI model 104 may be configured to transfer, based on the scheduled ER service 126, the first set of user-related data to the second AI model 112 associated with the second electronic healthcare system 116. For example, the first AI model 104 may transfer the first set of user-related data to the fourth AI model 124 based on the sync-up between the first AI model 104 and the fourth AI model 124. Details of the transfer of the first set of user-related data are further provided, for example, at 616 in FIG. 6B.

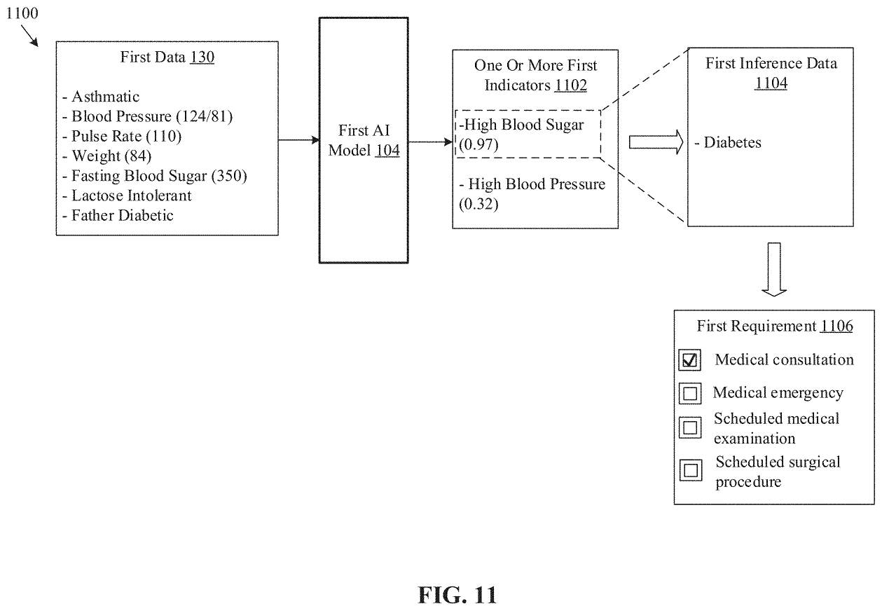

FIG. 11 is a diagram that depicts determination of an exemplary first requirement by use of a first AI model, in accordance with an embodiment of the disclosure. FIG. 11 is explained in conjunction with elements from FIGS. 1, 2, 3, 4A, 4B, 5A, 5B, 6A, 6B, 7A, 7B, 8, 9, 10A and 10B. With reference to FIG. 11, there is shown a diagram 1100. The diagram 1100 may include the first AI model 104.

The system 102 may apply the first AI model 104 on the first data 130 to compute one or more first indicators 1102. In an exemplary scenario, the first data 130 may include the historical health data of the user 108. For example, the user 108 may be asthmatic. The first data 130 may further include the set of sensor data. For example, the set of sensor data may include the recorded blood pressure measurements, including a current blood pressure measurement (such as 124/81 mmHg). The set of sensor data may include the recorded pulse rate measurement (such as a current pulse rate of 84 bpm). The set of sensor data may further include the weight of the user 108 and a current fasting blood sugar measurement (for example, 350). The first data 130 may further include the PFSH data associated with the user 108. For example, the PFSH data may include that the user 108 may be lactose intolerant and a parent (such as a father) of the user 108 may have a history of diabetes. The system 102 may input the first data 130 to the first AI model 104.

The first AI model 104 may compute the one or more first indicators 1102 based on the input first data 130. In an exemplary scenario, the one or more first indicators 1102 may include a first indicator corresponding to “high blood sugar” with the confidence score of “0.97”. The first AI model 104 may determine the first indicator corresponding to “high blood sugar” based on the fasting blood sugar measurement value of 350 that may be a large deviation from the reference value of the fasting blood sugar measurement. The one or more first indicators 1102 may include a second indicator corresponding to “high blood pressure” with the confidence score of “0.32”. For example, the first AI model 104 may determine the second indicator corresponding to “high blood pressure” based on the blood pressure measurement value of “124/81 mmHg”, that may be a small deviation from the reference value of the blood pressure measurement value of “120/80 mmHg”. Since the confidence score corresponding to the second indicator (“high blood pressure”) is “0.32”, the second indicator may be ignored.

Based on the first indicator corresponding to the high blood sugar, the system 102 may generate first inference data 1104. The first inference data 1104 may include the one or more labels or tags associated with the cause of the deviation in the health condition. For example, the first inference data 1104 may include the cause as “diabetes”. The system 102 may further determine a first requirement 1106 based on the first inference data 1104. In some embodiments, the first requirement 1106 may correspond to a medical consultation, a medical emergency, a scheduled medical examination, or a scheduled surgical procedure. Based on the determination that the first inference data 1104 is a non-emergency condition, the system 102 may determine the first requirement 1106 as “medical consultation”. Based on a preference of the user 108, the system 102 may book the medical consultation with a healthcare center, such as the first healthcare center 114.

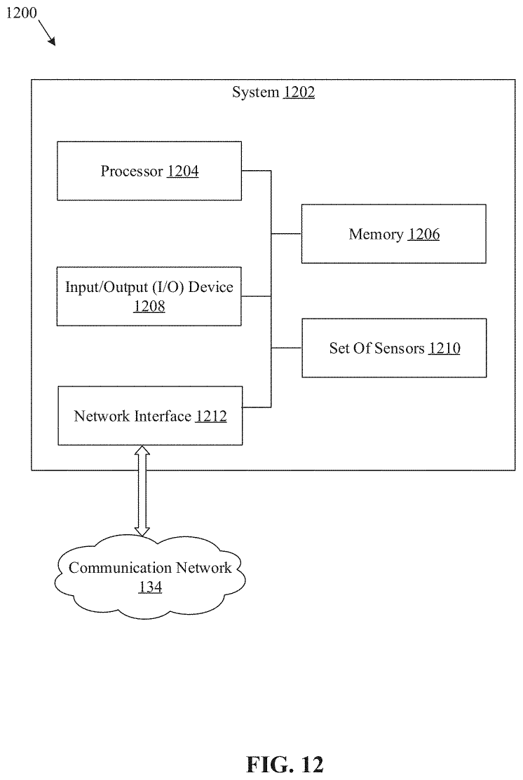

FIG. 12 is a block diagram of a system that enables artificial intelligence (AI)-based access to healthcare services, in accordance with an embodiment of the disclosure. FIG. 12 is explained in conjunction with elements from FIGS. 1, 2, 3, 4A, 4B, 5A, 5B, 6A, 6B, 7A, 7B, 8, 9, 10A, 10B, and 11. With reference to FIG. 12, there is shown a block diagram 1200 of a system 1202 that may be similar to the system 102 of FIG. 1. The system 1202 may include a processor 1204, a memory 1206, an input/output (I/O) device 1208, a set of sensors 1210, and a network interface 1212. The set of sensors 1210 may be similar to the set of sensors 132 of FIG. 1. Hence, the description of the set of sensors 1210 is omitted here for the sake of brevity.

The processor 1204 may include suitable logic, circuitry, and/or interfaces that may be configured to execute a set of instructions stored in the memory 1206. The processor 1204 may be configured to execute program instructions associated with different operations to be executed by the system 102. For example, some of the operations may include the reception of the collected first data 130, the application of the first AI model 104 on the collected first data 130 to compute the one or more first indicators, the generation of the first inference data, and the determination of the first requirement for which the user may be required to visit the first healthcare center 114. The processor 1204 may be further configured to determine the first set of user-related data and transfer the determined first set of user-related data to the first electronic healthcare system 110. The processor 1204 may be implemented based on a number of processor technologies known in the art. Examples of the processor technologies may include, but are not limited to, a Central Processing Unit (CPU), X86-based processor, a Reduced Instruction Set Computing (RISC) processor, an Application-Specific Integrated Circuit (ASIC) processor, a Complex Instruction Set Computing (CISC) processor, a Graphical Processing Unit (GPU), and other processors.

The memory 1206 may include suitable logic, circuitry, and interfaces that may be configured to store the one or more instructions to be executed by the processor 1204. The memory 1206 may be configured to store the collected first data 130, the one or more first indicators, the first inference data, the first requirement, and the first set of user-related data. The memory 1206 may be further configured to store the set of presentation data 210 and the medical data 214. The memory 1206 may further store the second inference data, the second requirement, and the second set of user-related data. Examples of implementation of the memory 1206 may include, but are not limited to, Random Access Memory (RAM), Read Only Memory (ROM), Electrically Erasable Programmable Read-Only Memory (EEPROM), Hard Disk Drive (HDD), a Solid-State Drive (SSD), a CPU cache, and/or a Secure Digital (SD) card.

The I/O device 1208 may include suitable logic, circuitry, and interfaces that may be configured to receive an input the user 108 and provide an output based on the received input. For example, the input may correspond to the request to share the data portion of the collected first data 130 with the first AI model 104. The input may further correspond to the first input including the first selection of the first healthcare center of the one or more healthcare centers and the second selection of the schedule for the appointment with the first healthcare center 114. The output may include, for example, the recommendations one or more healthcare centers. The I/O device 1208 which may include various input and output devices, may be configured to communicate with the processor 1204. Examples of the I/O device 1208 may include, but are not limited to, a touch screen, a keyboard, a mouse, a joystick, a microphone, a display device, and a speaker.

The network interface 1212 may include suitable logic, circuitry, interfaces, and/or code that may be configured to facilitate communication between the processor 1204, the first electronic healthcare system 110, the second electronic healthcare system 116, the third electronic healthcare system 122, and the server 128. The network interface 1212 may be implemented by use of various known technologies to support wired or wireless communication of the system 102 with the communication network 134. The network interface 1212 may include, but is not limited to, an antenna, a radio frequency (RF) transceiver, one or more amplifiers, a tuner, one or more oscillators, a digital signal processor, a coder-decoder (CODEC) chipset, a subscriber identity module (SIM) card, or a local buffer circuitry. The network interface 1212 may be configured to communicate via wireless communication with networks, such as the Internet, an Intranet or a wireless network, such as a cellular telephone network, a wireless local area network (LAN), and a metropolitan area network (MAN). The wireless communication may be configured to use one or more of a plurality of communication standards, protocols and technologies, such as Global System for Mobile Communications (GSM), Enhanced Data GSM Environment (EDGE), wideband code division multiple access (W-CDMA), Long Term Evolution (LTE), code division multiple access (CDMA), time division multiple access (TDMA), Bluetooth, Wireless Fidelity (Wi-Fi) (such as IEEE 802.11a, IEEE 802.11b, IEEE 802.11g or IEEE 802.11n), voice over Internet Protocol (VoIP), light fidelity (Li-Fi), Worldwide Interoperability for Microwave Access (Wi-MAX), a protocol for email, instant messaging, and a Short Message Service (SMS).

The functions or operations executed by the system 102, as described in FIG. 1, may be performed by the processor 1204. Operations executed by the processor 1204 are described in detail, for example, in FIGS. 2, 3, FIGS. 4A, 4B, 5A, 5B, 6A, 6B, 7A, 7B, 8, 9, 10A, 10B and 11.

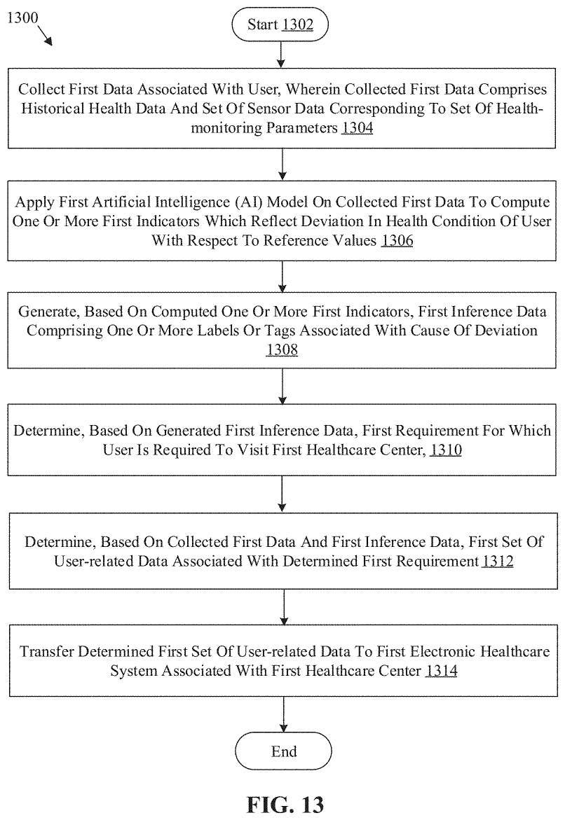

FIG. 13 is a flowchart that illustrates an exemplary method for artificial intelligence (AI)-enabled access to healthcare services, in accordance with an embodiment of the disclosure. FIG. 13 is described in conjunction with elements from FIGS. 1, 2, 3, 4A, 4B, 5A, 5B, 6A, 6B, 7A, 7B, 8, 9, 10A, 10B, 11 and 12. With reference to FIG. 13, there is shown a flowchart 1300. The exemplary method of the flowchart 1300 may be executed by any computing system, for example, by the system 102 of FIG. 1 or the processor 1204 of FIG. 12. The exemplary method of the flowchart 1300 may start at 1302 and proceed to 1304.

At 1304, the first data 130 associated with the user 108 may be collected. In accordance with an embodiment, the processor 1204 may be configured to collect the first data 130 associated with the user 108. The collected first data 130 may include the historical health data and the set of sensor data corresponding to the set of health-monitoring parameters. Details of the collection of the first data 130 are further provided, for example, in FIG. 2.

At 1306, the first AI model 104 may be applied on the collected first data 130 to compute the one or more first indicators, which may reflect the deviation in the health condition of the user 108 with respect to reference values. In accordance with an embodiment, the processor 1204 may apply the first AI model 104 on the collected first data 130 to compute the one or more first indicators. The one or more first indicators may reflect the deviation in the health condition of the user 108 with respect to reference values. Details of the computation of the one or more first indicators are further provided, for example, in FIG. 4A.

At 1308, based on the computed one or more first indicators, the first inference data, that may include the one or more labels or tags associated with the cause of the deviation, may be generated. In accordance with an embodiment, the processor 1204 may be configured to generate the first inference data, that may include the one or more labels or tags associated with the cause of the deviation, based on the computed one or more first indicators. Details of the generation of the first inference data are further provided, for example, in FIG. 4A.

At 1310, the first requirement, for which the user 108 may be required to visit the first healthcare center 114 may be determined, based on the generated first inference data. In accordance with an embodiment, the processor 1204 may be configured to determine the first requirement, for which the user 108 may be required to visit the first healthcare center 114, based on the generated first inference data. Details of the determination of the first requirement are further provided, for example, in FIG. 4A.

At 1312, the first set of user-related data associated with the determined first requirement may be determined, based on the collected first data 130 and the first inference data. In accordance with an embodiment, the processor 1204 may be configured to determine the first set of user-related data associated with the determined first requirement, based on the collected first data 130 and the first inference data. Details of the determination of the first set of user-related data are further provided, for example, in FIG. 4B.

At 1314, the determined first set of user-related data may be transferred to the first electronic healthcare system 110 associated with the first healthcare center 114. In accordance with an embodiment, the processor 1204 may be configured to transfer the determined first set of user-related data to the first electronic healthcare system 110 associated with the first healthcare center 114. Details of the transfer of the first set of user-related data are further provided, for example, in FIG. 4B. Control may pass to end.

Although the flowchart 1300 illustrates discrete operations, such as 1304, 1306, 1308, 1310, 1312, and 1314 the disclosure is not so limited. Accordingly, in certain embodiments, such discrete operations may be further divided into additional operations, combined into fewer operations, or eliminated, depending on the particular implementation without detracting from the essence of the disclosed embodiments.

Various embodiments of the disclosure may provide a non-transitory computer-readable medium and/or storage medium having stored thereon, computer-executable instructions executable by a machine and/or a computer in a system (for example the system 102). The instructions may cause the machine and/or computer in the system (for example, the system 102) to perform operations that may include collection of the first data 130 associated with the user 108. The collected first data 130 may include the historical health data and the set of sensor data corresponding to the set of health-monitoring parameters. The operations may further include application of the first AI model 104 on the collected first data 130 to compute the one or more first indicators which may reflect the deviation in the health condition of the user 108 with respect to reference values. The operations may further include generation, based on the computed one or more first indicators, the first inference data that may include the one or more class labels or tags associated with the cause of the deviation. The operations may further include determination, based on the generated first inference data, the first requirement for which the user 108 may be required to visit the first healthcare center 114. The operations may further include determination, based on the collected first data and the first inference data, the first set of user-related data associated with the determined first requirement. The operations may further include transfer of the determined first set of user-related data to the first electronic healthcare system 110 associated with the first healthcare center 114.

Exemplary aspects of the disclosure may include a system (such as the system 102) that may include a processor (such as the processor 1204). The processor 1204 may be configured to collect the first data 130 associated with the user 108. The collected first data 130 may include the historical health data and the set of sensor data corresponding to the set of health-monitoring parameters. The processor 1204 may be further configured to apply the first AI model 104 on the collected first data 130 to compute the one or more first indicators which may reflect the deviation in the health condition of the user 108 with respect to reference values. The processor 1204 may be further configured to generate, based on the computed one or more first indicators, the first inference data that may include the one or more class labels or tags associated with the cause of the deviation. The processor 1204 may be further configured to determine, based on the generated first inference data, the first requirement for which the user 108 may be required to visit the first healthcare center 114. The processor 1204 may be further configured to determine, based on the collected first data and the first inference data, the first set of user-related data associated with the determined first requirement. The processor 1204 may be further configured to transfer the determined first set of user-related data to the first electronic healthcare system 110 associated with the first healthcare center 114.

In accordance with an embodiment, the collected first data 130 may further include the past, family, and social history (PFSH) data, and the collaborative filtered data that may include health-related datapoints associated with the defined population, the specific set of geographies, the specific demography, or the viral infection or outbreak within the defined population.

In accordance with an embodiment, the collected first data 130 may further include the appointment schedule for the set of medical or health interventions at one or more healthcare centers, including the first healthcare center 114. The first requirement may be determined based on the appointment schedule.

In accordance with an embodiment, the set of health-monitoring parameters may be associated with at least one of the known health condition of the user 108, one or more medical interventions that the user 108 received in past, or one or more comorbidities associated with the user 108.

In accordance with an embodiment, the processor 1204 may be further configured to determine the current location of the user 108. The processor 1204 may be further configured to determine, by using the first AI model 104, the one or more recommendations that may include one or more healthcare centers associated with the determined first requirement. The processor 1204 may be further configured to control the user device 106 associated with the user 108 to display the determined one or more recommendations. The one or more healthcare centers may be within the threshold distance from the current location.

In accordance with an embodiment, the processor 1204 may be further configured to receive, via the user device 106, the first input. The first input may include the first selection of the first healthcare center 114 of the one or more healthcare centers, and the second selection of the schedule for the appointment with the first healthcare center 114. The processor 1204 may be further configured to schedule the visit to the first healthcare center 114, based on the received first input. The first set of user-related data may be transferred to the first electronic healthcare system 110 based on the scheduling.

In accordance with an embodiment, the processor 1204 may be further configured to transmit a request to the first electronic healthcare system 110 to authorize the virtual reality (VR)-based consultation session, based on the determination that the current location of the user 108 is different from the location of the first healthcare center 114. The processor 1204 may be further configured to receive an authorization to the transmitted request and establish, based on the received authorization, the VR-based consultation session between the user device 106 and the wearable electronic device worn by the medical practitioner 212 at the first healthcare center 114. While the VR-based consultation session may be active, the determined first set of user-related data may be transferred to the wearable electronic device.

In accordance with an embodiment, the processor 1204 may be further configured to generate the set of presentation data 210 by applying the second AI model 112 on the transferred first set of user-related data. The set of presentation data 210 may include datapoints which may be required by the medical practitioner 212 associated with the first healthcare center 114 to assess the current health condition of the user 108 and to service the determined first requirement. The processor 1204 may be further configured to control the display device associated with the first healthcare center 114 to display the generated set of presentation data 210.

In accordance with an embodiment, the second AI model 112 may be the conversational AI hosted on the first electronic healthcare system 110 and may be associated with the first healthcare center 114.

In accordance with an embodiment, the processor 1204 may be further configured to detect the presence of the user 108 at the first healthcare center 114. The processor 1204 may be further configured to collect, based on the detection, the medical data 214 associated with the medical attention received by the user 108 at the first healthcare center 114 as part of the determined first requirement and update the first AI model 104 based on the collected medical data 214.

In accordance with an embodiment, the processor 1204 may be further configured to apply the first AI model 104 on the collected medical data 214 and the collected first data 130 to generate second inference data. The processor 1204 may be further configured to determine, based on the generated second inference data, the second requirement for which the user 108 may be required to visit the second healthcare center 120 which is different from the first healthcare center 114. The processor 1204 may be further configured to determine, based on the collected first data 130, the collected medical data 214, and the second inference data, the second set of user-related data which may be associated with the determined second requirement and may be required by the second electronic healthcare system 116 associated with the second healthcare center 120. The processor 1204 may be further configured to transfer the determined second set of user-related data to the second electronic healthcare system 116.

In accordance with an embodiment, the processor 1204 may be further configured to determine the second healthcare center 120 based on the current location of the user 108 and the determination that the determined first requirement may correspond to the medical emergency. The processor 1204 may be further configured to schedule the ER service 126. The processor 1204 may be further configured to transfer, based on the scheduled ER service 126, the first set of user-related data to the second electronic healthcare system 116 associated with the second healthcare center 120.

In accordance with an embodiment, the processor 1204 may be further configured to transmit the alert notification to the one or more devices registered for receiving the alert notification, based on the determination that the first requirement may correspond to the medical emergency.

In accordance with an embodiment, the processor 1204 may be further configured to receive, by the user device 106, the request to share the data portion of the collected first data 130 with the first AI model 104. The processor 1204 may be further configured to create the encrypted session between the first AI model 104 and the user device 106, based on the request. The processor 1204 may be further configured to transfer, while the encrypted session may be active, the data portion of the collected first data 130 to the first AI model 104. The processor 1204 may be further configured to store the transferred data portion in the encrypted form on the datastore.

The present disclosure may be realized in hardware, or a combination of hardware and software. The present disclosure may be realized in a centralized fashion, in at least one computer system, or in a distributed fashion, where different elements may be spread across several interconnected computer systems. A computer system or other apparatus adapted to carry out the methods described herein may be suited. A combination of hardware and software may be a general-purpose computer system with a computer program that, when loaded and executed, may control the computer system such that it carries out the methods described herein. The present disclosure may be realized in hardware that includes a portion of an integrated circuit that also performs other functions.

The present disclosure may also be embedded in a computer program product, which includes all the features that enable the implementation of the methods described herein, and which when loaded in a computer system is able to carry out these methods. Computer program, in the present context, means any expression, in any language, code or notation, of a set of instructions intended to cause a system with information processing capability to perform a particular function either directly, or after either or both of the following: a) conversion to another language, code or notation; b) reproduction in a different material form.

While the present disclosure is described with reference to certain embodiments, it will be understood by those skilled in the art that various changes may be made, and equivalents may be substituted without departure from the scope of the present disclosure. In addition, many modifications may be made to adapt a particular situation or material to the teachings of the present disclosure without departure from its scope. Therefore, it is intended that the present disclosure is not limited to the particular embodiment disclosed, but that the present disclosure will include all embodiments that fall within the scope of the appended claims.

What is claimed is:

1. A method, comprising:
collecting first data associated with a user,
wherein the collected first data comprises historical health data and a set of sensor data corresponding to a set of health-monitoring parameters;
applying a first Artificial Intelligence (AI) model on the collected first data to compute one or more first indicators which reflect a deviation in a health condition of the user with respect to reference values;
generating, based on the computed one or more first indicators, first inference data comprising one or more labels or tags associated with a cause of the deviation;
determining, based on the generated first inference data, a first requirement for which the user is required to visit a first healthcare center;
determining, based on the collected first data and the first inference data, a first set of user-related data associated with the determined first requirement; and
transferring the determined first set of user-related data to a first electronic healthcare system associated with the first healthcare center.

2. The method according to claim 1, wherein the collected first data further comprises of:
past, family, and social history (PFSH) data, and
collaborative filtered data that includes health-related datapoints associated with a defined population, a specific set of geographies, a specific demography, or a viral infection or outbreak within the defined population.

3. The method according to claim 1, wherein the collected first data further comprises an appointment schedule for a set of medical or health interventions at one or more healthcare centers, including the first healthcare center, and
wherein the first requirement is determined based on the appointment schedule.

4. The method according to claim 1, wherein the set of health-monitoring parameters is associated with at least one of a known health condition of the user, one or more medical interventions that the user received in past, or one or more comorbidities associated with the user.

5. The method according to claim 1, further comprising:
determining a current location of the user;
determining, by using the first AI model, one or more recommendations comprising one or more healthcare centers associated with the determined first requirement; and
controlling a user device associated with the user to display the determined one or more recommendations,
wherein the one or more healthcare centers are within a threshold distance from the current location.

6. The method according to claim 5, further comprising:
receiving, via the user device, a first input comprising:
a first selection of the first healthcare center of the one or more healthcare centers, and
a second selection of a schedule for an appointment with the first healthcare center; and
scheduling the visit to the first healthcare center, based on the received first input,
wherein the first set of user-related data is transferred to the first electronic healthcare system based on the scheduling.

7. The method according to claim 1, further comprising:
transmitting a request to the first electronic healthcare system to authorize a virtual reality (VR)-based consultation session, based on a determination that a current location of the user is different from a location of the first healthcare center;
receiving an authorization to the transmitted request; and
establishing, based on the received authorization, the VR-based consultation session between a user device and a wearable electronic device worn by a medical practitioner at the first healthcare center,
wherein, while the VR-based consultation session is active, the determined first set of user-related data is transferred to the wearable electronic device.

8. The method according to claim 1, further comprising:
generating a set of presentation data by applying a second AI model on the transferred first set of user-related data,
wherein the set of presentation data includes datapoints which are required by a medical practitioner associated with the first healthcare center to assess a current health condition of the user and to service the determined first requirement; and
controlling a display device associated with the first healthcare center to display the generated set of presentation data.

9. The method according to claim 8, wherein the second AI model is a conversational AI hosted on the first electronic healthcare system and is associated with the first healthcare center.

10. The method according to claim 1, further comprising:
detecting a presence of the user at the first healthcare center; and
collecting, based on the detection, medical data associated with a medical attention received by the user at the first healthcare center as part of the determined first requirement; and
updating the first AI model based on the collected medical data.

11. The method according to claim 10, further comprising:
applying the first AI model on the collected medical data and the collected first data to generate second inference data;
determining, based on the generated second inference data, a second requirement for which the user is required to visit a second healthcare center which is different from the first healthcare center;
determining, based on the collected first data, the collected medical data, and the second inference data, a second set of user-related data which is associated with the determined second requirement and is required by a second electronic healthcare system associated with the second healthcare center; and
transferring the determined second set of user-related data to the second electronic healthcare system.

12. The method according to claim 1, further comprising:
determining a second healthcare center based on a current location of the user and a determination that the determined first requirement corresponds to a medical emergency; and
scheduling an emergency response (ER) service; and
transferring, based on the scheduled ER service, the first set of user-related data to a second electronic healthcare system associated with the second healthcare center.

13. The method according to claim 1, further comprising transmitting an alert notification to one or more devices registered for receiving the alert notification, based on a determination that the first requirement corresponds to a medical emergency.

14. The method according to claim 1, further comprising:
receiving, by a user device, a request to share a data portion of the collected first data with the first AI model;
creating an encrypted session between the first AI model and the user device, based on the request;
transferring, while the encrypted session is active, the data portion of the collected first data to first AI model; and
storing the transferred data portion in an encrypted form on a datastore.

15. A system, comprising:
a processor configured to:
collect first data associated with a user,
wherein the collected first data comprises historical health data and a set of sensor data corresponding to a set of health-monitoring parameters;
apply a first Artificial Intelligence (AI) model on the collected first data to compute one or more first indicators which reflect a deviation in a health condition of the user with respect to reference values;
generate, based on the computed one or more first indicators, first inference data comprising one or more labels or tags associated with a cause of the deviation;
determine, based on the generated first inference data, a first requirement for which the user is required to visit a first healthcare center;
determine, based on the collected first data and the first inference data, a first set of user-related data associated with the determined first requirement; and
transfer the determined first set of user-related data to a first electronic healthcare system associated with the first healthcare center.

16. The system according to claim 15, wherein the collected first data further comprises an appointment schedule for a set of medical or health interventions at one or more healthcare centers, including the first healthcare center, and
wherein the first requirement is determined based on the appointment schedule.

17. The system according to claim 15, wherein the processor is further configured to:
determine a current location of the user;
determine, by using the first AI model, one or more recommendations comprising one or more healthcare centers associated with the determined first requirement;
control a user device associated with the user to display the determined one or more recommendations,
wherein the one or more healthcare centers are within a threshold distance from the current location;
receive, via the user device, a first input comprising:
a first selection of the first healthcare center of the one or more healthcare centers, and
a second selection of a schedule for an appointment with the first healthcare center; and
schedule the visit to the first healthcare center, based on the received first input,
wherein the first set of user-related data is transferred to the first electronic healthcare system based on the scheduling.

18. The system according to claim 15, wherein the processor is further configured to:
generate a set of presentation data by application of a second AI model on the transferred first set of user-related data,
wherein the set of presentation data includes datapoints which are required by a medical practitioner associated with the first healthcare center to assess a current health condition of the user and to service the determined first requirement; and
control a display device associated with the first healthcare center to display the generated set of presentation data.

19. The system according to claim 18, wherein the second AI model is a conversational AI hosted on the first electronic healthcare system and is associated with the first healthcare center.

20. A non-transitory computer-readable storage medium configured to store instructions that, when executed by a computer in a system, causes the computer in the system to perform operations, the operations comprising:
collecting first data associated with a user,
wherein the collected first data comprises historical health data and a set of sensor data corresponding to a set of health-monitoring parameters;
applying a first Artificial Intelligence (AI) model on the collected first data to compute one or more first indicators which reflect a deviation in a health condition of the user with respect to reference values;
generating, based on the computed one or more first indicators, first inference data comprising one or more class labels or tags associated with a cause of the deviation;
determining, based on the generated first inference data, a first requirement for which the user is required to visit a first healthcare center;
determining, based on the collected first data and the first inference data, a first set of user-related data associated with the determined first requirement; and
transferring the determined first set of user-related data to a first electronic healthcare system associated with the first healthcare center.
